(19) United States
(12) Patent Application Publication
(10) Pub. No.: US 2025/0086799 A1
(43) Pub. Date: Mar. 13, 2025

(54) INFORMATION PROCESSING APPARATUS, ENDOSCOPE APPARATUS, INFORMATION PROCESSING METHOD, AND PROGRAM

(71) Applicant: FUJIFILM CORPORATION, Tokyo (JP)

(72) Inventors: Yohei KUBO, Kanagawa (JP); Manabu MIYAMOTO, Kanagawa (JP); Masahiro KUBO, Kanagawa (JP)

(21) Appl. No.: 18/956,007

(22) Filed: Nov. 22, 2024

Related U.S. Application Data
(63) Continuation of application No. PCT/JP2023/018160, filed on May 15, 2023.

(30) Foreign Application Priority Data
Jun. 9, 2022 (JP) 2022-093907

(51) Int. Cl. G06T 7/00; G06T 7/70

(52) U.S. Cl. CPC G06T 7/0014; G06T 7/70; G06T 2207/10068

(57) ABSTRACT

An information processing apparatus includes a processor. The processor is configured to: acquire multiple first endoscopic examination images from storage archiving the multiple first endoscopic examination images, the multiple endoscopic examination images having been obtained by imaging multiple sites in a first endoscopic examination which is a previous or earlier endoscopic examination; and cause a display apparatus to display at least one first endoscopic examination image satisfying a specific condition from among the multiple first endoscopic examination images as a reference image in a period of carrying out a second endoscopic examination which is a current endoscopic examination.

### CROSS-REFERENCE TO RELATED APPLICATIONS

This application is a continuation application of International Application No. PCT/JP2023/018160, filed May 15, 2023, the disclosure of which is incorporated herein by reference in its entirety. Further, this application claims priority from Japanese Patent Application No. 2022-093907, filed Jun. 9, 2022, the disclosure of which is incorporated herein by reference in its entirety.

### BACKGROUND

### 1. Technical Field

The technology of the present disclosure relates to an information processing apparatus, an endoscope apparatus, an information processing method, and a program.

### 2. Related Art

JP6284439B discloses a medical information processing system provided with an endoscope system for performing endoscopic examinations on a subject and an examination information management system that manages past examination information. The examination information management system includes: an examination information storage unit storing past examination information; an examination information extraction unit that extracts past examination information about a subject; and a transmission unit that transmits the extracted past examination information about the subject to the endoscope system. The endoscope system is provided with: a reception unit that receives past examination information about a subject from the examination information management system; an image acquisition unit that acquires an observation image of the subject captured by an imaging apparatus; a display control unit that causes a display apparatus to display the observation image acquired by the image acquisition unit and information pertaining to an observation site included in the examination information received by the reception unit; a completion information accepting unit that accepts site observation completion information indicating that the observation of the observation site corresponding to the information displayed on the display apparatus has been completed; and a site information registration unit that registers the site observation completion information. When the completion information accepting unit accepts the site observation completion information, the site information registration unit registers the site observation completion information in association with information pertaining to the observation site, and when the site information registration unit registers the site observation completion information, the display control unit causes the display apparatus to display information pertaining to the observation site to be observed next.

WO2019/049451A discloses a video processor provided with: a time measurement unit that measures the elapsed time from a reference timing in an endoscopic examination; a storage unit storing, in association with each other, a first endoscopic observation image acquired in a first endoscopic examination and an elapsed time from the reference timing in the first endoscopic examination at the acquisition time of the first endoscopic observation image; and a display control unit that carries out control so as to simultaneously display a second endoscopic observation image acquired in a second endoscopic examination performed after the first endoscopic examination and the first endoscopic observation image stored in the storage unit in association with the same elapsed time as the elapsed time from the reference timing in the second endoscopic examination at the acquisition time of the second endoscopic observation image.

### SUMMARY

One embodiment according to the technology of the present disclosure provides an information processing apparatus, an endoscope apparatus, an information processing method, and a program enabling a user to understand changes in a site deemed the main examination target of endoscopic examination.

A first aspect according to the technology of the present disclosure is an information processing apparatus including a processor configured to: acquire multiple first endoscopic examination images from storage archiving the multiple first endoscopic examination images, the multiple endoscopic examination images having been obtained by imaging multiple sites in a first endoscopic examination which is a previous or earlier endoscopic examination; and cause a display apparatus to display at least one first endoscopic examination image satisfying a specific condition from among the multiple first endoscopic examination images as a reference image in a period of carrying out a second endoscopic examination which is a current endoscopic examination.

A second aspect according to the technology of the present disclosure is the information processing apparatus according to the first aspect, wherein the processor is configured to cause the display apparatus to display the reference image in a timeout phase performed during the period of carrying out the second endoscopic examination.

A third aspect according to the technology of the present disclosure is the information processing apparatus according to the second aspect, wherein the processor is configured to cause the display apparatus to display first information obtained during a period of carrying out the first endoscopic examination, in a timeout phase performed during the period of carrying out the second endoscopic examination.

A fourth aspect according to the technology of the present disclosure is the information processing apparatus according to any one of the first to third aspects, wherein the processor is configured to acquire second information from an apparatus storing the second information, the second information being required in a timeout performed during the period of carrying out the second endoscopic examination, and cause the display apparatus to display the acquired second information.

A fifth aspect according to the technology of the present disclosure is the information processing apparatus according to the fourth aspect, wherein the processor is configured to store the second information in the storage upon completion of the timeout performed during the period of carrying out the second endoscopic examination.

A sixth aspect according to the technology of the present disclosure is the information processing apparatus according to any one of the first to fifth aspects, wherein information obtained in a timeout of the first endoscopic examination is associated with the first endoscopic examination images.

A seventh aspect according to the technology of the present disclosure is the information processing apparatus according to any one of the first to sixth aspects, wherein information obtained in a timeout of the second endoscopic examination is associated with a second endoscopic examination image obtained by imaging in the second endoscopic examination.

An eighth aspect according to the technology of the present disclosure is the information processing apparatus according to any one of the first to seventh aspects, wherein the specific condition is a condition stipulating that a selection has been made according to an instruction accepted by an accepting apparatus.

A ninth aspect according to the technology of the present disclosure is the information processing apparatus according to any one of the first to seventh aspects, wherein the specific condition is a condition stipulating that a selection has been made by performing image recognition processing on the multiple first endoscopic examination images and/or information processing on metadata of the multiple first endoscopic examination images.

A 10th aspect according to the technology of the present disclosure is the information processing apparatus according to any one of the first to seventh aspects, wherein the specific condition is a condition stipulating that a selection has been made by performing image recognition processing on the multiple first endoscopic examination images and/or information processing on metadata of the multiple first endoscopic examination images, and that a selection has been made according to an instruction accepted by an accepting apparatus.

An 11th aspect according to the technology of the present disclosure is the information processing apparatus according the ninth or 10th aspect, wherein the metadata includes endoscopic examination information obtained during the period of carrying out the first endoscopic examination.

A 12th aspect according to the technology of the present disclosure is the information processing apparatus according to the 11th aspect, wherein the endoscopic examination information includes information obtained in a timeout of the first endoscopic examination.

A 13th aspect according to the technology of the present disclosure is the information processing apparatus according to any one of the first to 12th aspects, wherein the processor is configured to perform first notification processing to provide a notification when a location corresponding to a characteristic portion in the reference image is shown in a second endoscopic examination image obtained by imaging in the second endoscopic examination.

A 14th aspect according to the technology of the present disclosure is the information processing apparatus according to the 13th aspect, wherein the first notification processing includes processing to change a display appearance of the reference image.

A 15th aspect according to the technology of the present disclosure is the information processing apparatus according to any one of the first to 14th aspects, wherein the processor is configured to change a display appearance of the reference image according to a positional relationship between a characteristic portion in the reference image and a location which corresponds to the characteristic portion and which is shown in a second endoscopic examination image obtained by imaging in the second endoscopic examination.

A 16th aspect according to the technology of the present disclosure is the information processing apparatus according to the 15th aspect, wherein the processor is configured to perform second notification processing to provide a notification when the positional relationship is a predetermined positional relationship.

A 17th aspect according to the technology of the present disclosure is the information processing apparatus according to any one of the first to 16th aspects, wherein the processor is configured to perform assistance processing to assist with making imaging conditions of the second endoscopic examination consistent with imaging conditions of the first endoscopic examination on the basis of the reference image and a second endoscopic examination image obtained by imaging in the second endoscopic examination.

An 18th aspect according to the technology of the present disclosure is the information processing apparatus according to the 17th aspect, wherein the assistance processing includes output processing to output assistance information required to make the imaging conditions of the second endoscopic examination consistent with the imaging conditions of the first endoscopic examination.

A 19th aspect according to the technology of the present disclosure is the information processing apparatus according to the 18th aspect, wherein the assistance information is derived on the basis of a result of comparing a first characteristic portion shown in the reference image with a second characteristic portion shown in the second endoscopic examination image.

A 20th aspect according to the technology of the present disclosure is the information processing apparatus according to any one of the first to 19th aspects, wherein the assistance processing includes third notification processing to provide a notification when the imaging conditions of the second endoscopic examination and the imaging conditions of the first endoscopic examination are matching.

A 21st aspect according to the technology of the present disclosure is the information processing apparatus according to any one of the first to 20th aspects, wherein the processor is configured to change the composition of the reference image according to a second endoscopic examination image obtained by imaging in the second endoscopic examination.

A 22nd aspect according to the technology of the present disclosure is the information processing apparatus according to any one of the first to 21st aspects, wherein the processor is configured to determine, on the basis of the reference image and a second endoscopic examination image obtained by imaging in the second endoscopic examination, whether or not a common lesion is shown in the reference image and the second endoscopic examination image.

A 23rd aspect according to the technology of the present disclosure is the information processing apparatus according to the 20th aspect, wherein the processor is configured to perform fourth notification processing to provide a notification when it is determined that a common lesion is shown in the reference image and the second endoscopic examination image and an instruction to confirm the determination result is accepted by an accepting apparatus, the notification indicating confirmation of the determination result.

A 24th aspect according to the technology of the present disclosure is the information processing apparatus according to any one of the first to 23rd aspects, wherein the processor is configured to generate lesion-related information when a common lesion is shown in the reference image and a second endoscopic examination image obtained by imaging in the second endoscopic examination, the lesion-related information pertaining to the lesion.

A 25th aspect according to the technology of the present disclosure is the information processing apparatus according to the 24th aspect, wherein the lesion-related information includes size-related information pertaining to the size of the lesion.

A 26th aspect according to the technology of the present disclosure is the information processing apparatus according to the 25th aspect, wherein the size-related information includes change-identifying information that can be used to identify change over time in the size.

A 27th aspect according to the technology of the present disclosure is the information processing apparatus according to the 26th aspect, wherein the change-identifying information is derived on the basis of the size of the lesion shown in the reference image and/or the size of the lesion shown in the second endoscopic examination image.

A 28th aspect according to the technology of the present disclosure is the information processing apparatus according to the 27th aspect, wherein the change-identifying information is derived on the basis of the size of the lesion shown in the reference image and/or the size of the lesion shown in the second endoscopic examination image, and the type of the lesion.

A 29th aspect according to the technology of the present disclosure is the information processing apparatus according to any one of the 24th to 28th aspects, wherein the lesion-related information includes information that can be used to identify the type of the lesion, information that can be used to identify the number of lesions, and/or information that can be used to identify the state of the lesion.

A 30th aspect according to the technology of the present disclosure is the information processing apparatus according to any one of the 24th to 29th aspects, wherein the lesion-related information is associated with the reference image showing the lesion and/or the second endoscopic examination image showing the lesion.

A 31st aspect according to the technology of the present disclosure is the information processing apparatus according to any one of the 24th to 30th aspects, wherein the processor is configured to create a report in which the lesion-related information, the reference image showing the lesion, and/or the second endoscopic examination image showing the lesion are recorded.

A 32nd aspect according to the technology of the present disclosure is the information processing apparatus according to any one of the first to 31st aspects, wherein the processor is configured to cause the display apparatus to display the reference image and a second endoscopic examination image obtained by imaging in the second endoscopic examination in a manner allowing for comparison.

A 33rd aspect according to the technology of the present disclosure is an endoscope apparatus including the information processing apparatus according to any one of the first to third aspects and an endoscope that images the multiple sites in the endoscopic examinations.

A 34th aspect according to the technology of the present disclosure is an information processing method including: acquiring multiple first endoscopic examination images from storage archiving the multiple first endoscopic examination images, the multiple first endoscopic examination images having been obtained by imaging multiple sites in a first endoscopic examination which is a previous or earlier endoscopic examination; and causing a display apparatus to display at least one first endoscopic examination image satisfying a specific condition from among the multiple first endoscopic examination images as a reference image in a period of carrying out a second endoscopic examination which is a current endoscopic examination.

A 35th aspect according to the technology of the present disclosure is a program causing a computer to execute a process including: acquiring multiple first endoscopic examination images from storage archiving the multiple first endoscopic examination images, the multiple first endoscopic examination images having been obtained by imaging multiple sites in a first endoscopic examination which is a previous or earlier endoscopic examination; and causing a display apparatus to display at least one first endoscopic examination image satisfying a specific condition from among the multiple first endoscopic examination images as a reference image in a period of carrying out a second endoscopic examination which is a current endoscopic examination.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example of an overall configuration of an endoscope system;

FIG. 3 is a conceptual diagram illustrating an example of an aspect in which the insertion part of an endoscope is inserted into the large intestine of a subject;

FIG. 4 is a block diagram illustrating an example of a hardware configuration of an endoscopic processing apparatus;

FIG. 5 is a block diagram illustrating an example of a hardware configuration of a control apparatus;

FIG. 6 is a block diagram illustrating an example of a hardware configuration of a server;

FIG. 7 is a block diagram illustrating an example of major functions of a processor of a control apparatus;

FIG. 8 is a block diagram illustrating an example of major functions of a processor of a server;

FIG. 9 is a conceptual diagram illustrating an example of processing details for causing a display apparatus to display an endoscopic image;

FIG. 10 is a conceptual diagram illustrating an example of an aspect in which a report is exchanged between a server and a control apparatus;

FIG. 11 is a conceptual diagram illustrating an example of the details of a report;

FIG. 12 is a conceptual diagram illustrating an example of an aspect in which a server transmits a report to an endoscope apparatus in response to a request from the endoscope apparatus;

FIG. 13 is a conceptual diagram illustrating an example of processing details for generating a past examination image screen and a subject information screen on the basis of a report;

FIG. 14 is a conceptual diagram illustrating an example of an aspect in which a display apparatus is caused to display subject-identifying information and an example of an aspect in which a touch panel display of a tablet terminal is caused to selectively display a past examination image screen and a subject information screen;

FIG. 15 is a conceptual diagram illustrating an example of an aspect in which an examination result image selected according to an image selection instruction is selected as a reference image and stored in a reference image storage area;

FIG. 16 is a conceptual diagram illustrating an example of an aspect in which information obtained during a second endoscopic examination timeout is recorded in a report and displayed on a touch panel display of a tablet terminal;

FIG. 17 is a conceptual diagram illustrating an example of an aspect in which an image recognition unit executes image recognition processing on a reference image and compares a characteristic portion extracted by the image recognition processing with an endoscopic image;

FIG. 18 is a conceptual diagram illustrating an example of an aspect in which a reference image is displayed on a past examination image screen when a reference image display instruction is accepted by an accepting apparatus and an example of an aspect in which a reference image is displayed in an enlarged manner on a past examination image screen when a corresponding characteristic portion is shown in an endoscopic image;

FIG. 19 is a conceptual diagram illustrating an example of an aspect in which assistance information is derived on the basis of an endoscopic image and a reference image, and the derived assistance information is displayed on a screen of a display apparatus;

FIG. 20 is a conceptual diagram indicating processing details in a case where the imaging conditions of a second endoscopic examination and the imaging conditions of a first endoscopic examination are matching;

FIG. 21 is a conceptual diagram illustrating an example of processing details for determining whether or not a common lesion is shown in an endoscopic image and a reference image, and providing a notification when a common lesion is shown in the endoscopic image and the reference image;

FIG. 22 is a conceptual diagram illustrating an example of processing details in a case where a determination result is confirmed to indicate that a common lesion is shown in an endoscopic image and a reference image;

FIG. 23 is a conceptual diagram illustrating an example of processing details for recording timeout information and an endoscopic image in association with each other in a report by including the timeout information in metadata of the endoscopic image, the timeout information having been obtained in a timeout performed within the period of carrying out a second endoscopic examination, and storing the report in NVM of a server;

FIG. 24 is a conceptual diagram illustrating an example of processing details for generating lesion-related information and including the generated lesion-related information in metadata;

FIG. 25 is a flowchart illustrating an example of the flow of endoscopic image display processing;

FIG. 26A is a flowchart illustrating an example of the flow of endoscope manipulation assistance processing;

FIG. 26B is a continuation of the flowchart illustrated in FIG. 26A;

FIG. 26C is a continuation of the flowchart illustrated in FIG. 26B;

FIG. 26D is a continuation of the flowchart illustrated in FIG. 26C;

FIG. 27 is a flowchart illustrating an example of the flow of report management processing;

FIG. 28 is a conceptual diagram illustrating an example of processing details for making the scale of a reference image consistent with the scale of an endoscopic image;

FIG. 29 is a conceptual diagram illustrating an example of processing details for making the composition of a reference image consistent with the composition of an endoscopic image;

FIG. 30 is a conceptual diagram illustrating an example of an aspect in which a reference image is selected by performing image recognition processing on multiple examination result images; and

FIG. 31 is a conceptual diagram illustrating an example of an aspect in which a reference image is selected on the basis of a result of performing information processing on multiple pieces of metadata corresponding to multiple examination result images.

### DETAILED DESCRIPTION

The following describes, in accordance with the attached drawings, examples of embodiments of an information processing apparatus, an endoscope apparatus, an information processing method, and a program according to the technology of the present disclosure.

First, terms used in the following description will be explained.

CPU is an abbreviation for “central processing unit”. GPU is an abbreviation for “graphics processing unit”. RAM is an abbreviation for “random access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electroluminescence”. CMOS is an abbreviation for “complementary metal-oxide-semiconductor”. CCD is an abbreviation for “charge-coupled device”. LAN is an abbreviation for “local area network”. WAN is an abbreviation for “wide area network”. AI is an abbreviation for “artificial intelligence”. BLI is an abbreviation for “blue light imaging”. LCI is an abbreviation for “linked color imaging”. In the embodiments herein, “matching” refers not only to perfectly matching but also to matching in the sense of including error which is generally acceptable in the technical field to which the technology of the present disclosure belongs and which does not contradict the gist of the technology of the present disclosure.

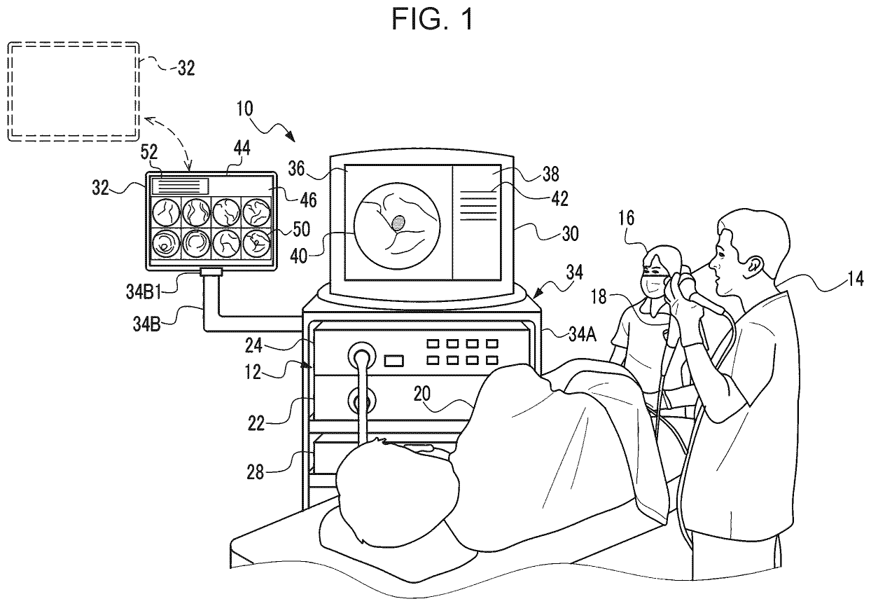

As illustrated by way of example in FIG. 1, an endoscope system 10 is provided with an endoscope apparatus 12. The endoscope apparatus 12 is used by a physician 14 in an endoscopic examination. Also, at least one auxiliary staff member 16 (a nurse, for example) assists the physician 14 in the endoscopic examination. In the following, the physician 14 and the auxiliary staff member 16 are also referred to as the “user” without an accompanying reference sign when it is not necessary to distinguish between them.

The endoscope apparatus 12 is provided with an endoscope (scope) and is used to perform examination and treatment inside the body of a subject 20 (a patient, for example) via the endoscope 18. The endoscope apparatus 12 is an example of an “endoscope apparatus” according to the technology of the present disclosure. The endoscope 18 is an example of an “endoscope” according to the technology of the present disclosure.

The endoscope 18 images the inside of the body of the subject 20 to thereby acquire and output an image illustrating the state inside the body. The example illustrated in FIG. 1 illustrates a situation in which the endoscope 18 is inserted into the body cavity from the anus of the subject 20. Note that in the example illustrated in FIG. 1, the endoscope 18 is inserted into the body cavity from the anus of the subject 20, but this is merely one example, and the endoscope 18 may also be inserted into the body cavity from the mouth or nostril of the subject 20 or from a perforation or the like. The location where the endoscope 18 is to be inserted is determined according to the type of endoscope 18, the operative technique, and the like.

In the following, an endoscopic examination performed on the subject 20 in the past is referred to as the “first endoscopic examination” for convenience. An endoscopic examination performed on the subject 20 in the past refers to the previous or earlier endoscopic examination performed on the subject 20. One example of the previous or earlier endoscopic examination performed on the subject 20 is the last endoscopic examination performed on the subject 20, but this is merely one example, and the previous or earlier endoscopic examination may also be multiple prior endoscopic examinations performed on the subject 20. In the following, the current endoscopic examination of the subject 20 is referred to as the “second endoscopic examination” for convenience. Also, in the following, the first endoscopic examination and the second endoscopic examination are simply referred to as the “endoscopic examination(s)” when it is not necessary to distinguish between them. The following description assumes that in each of the first endoscopic examination and the second endoscopic examination, multiple locations inside the body (the inner wall of the large intestine, for example) of the subject 20 are imaged by the endoscope 18. These multiple locations inside the body of the subject 20 are an example of “multiple sites” according to the technology of the present disclosure.

The endoscope apparatus 12 is provided with an endoscopic processing apparatus 22, a light source apparatus 24, a control apparatus 28, a display apparatus 30, and a tablet terminal 32. The endoscopic processing apparatus 22, light source apparatus 24, control apparatus 28, display apparatus 30, and tablet terminal 32 are installed in an arm-attached wagon 34. The arm-attached wagon 34 has a wagon 34A and an arm 34B. The wagon 34A has multiple platforms provided along the vertical direction, and from the lower platform to the upper platform, the control apparatus 28, the endoscopic processing apparatus 22, the light source apparatus 24, and the display apparatus 30 are installed.

The arm 34B is attached to the wagon 34A. The arm 34B extends in the horizontal direction from a side surface of the wagon 34A. A holder 34B1 is provided on the leading end part of the arm 34B. The holder 34B1 holds the tablet terminal 32 by clamping an end of the tablet terminal 32. The tablet terminal 32 can be removed from the holder 34B1 by releasing the clamping state of the holder 34B1. By having the arm 34B hold the tablet terminal 32, the tablet terminal 32 and the display apparatus 30 on top of the wagon 34A are arranged side by side.

The display apparatus 30 displays various information, including images. The display apparatus 30 may be a liquid crystal display or an EL display, for example. Multiple screens are arranged and displayed on the display apparatus 30. In the example illustrated in FIG. 1, screens 36 and 38 are illustrated as an example of the multiple screens.

An endoscopic image 40 is displayed on the screen 36. The endoscopic image 40 is an image acquired by imaging an observation target region with the endoscope 18 inside the body cavity of the subject 20. The observation target region may be the inner wall of the large intestine. The inner wall of the large intestine is merely one example, and the observation target region may also be the inner wall or outer wall of the small intestine, the duodenum, or another part of the stomach, or the like.

The endoscopic image 40 displayed on the screen 36 is one frame included in a dynamic image formed from multiple frames. That is, multiple frames of endoscopic images 40 are displayed on the screen 36 at a predetermined frame rate (30 frames per second or 60frames per second, for example).

Subject-identifying information 42 is displayed on the screen 38. The subject-identifying information 42 is information pertaining to the subject 20. The subject-identifying information 42 includes, for example, the name of the subject 20, the age of the subject 20, an identification number that can be used to identify the subject 20, and information to be aware of when performing a procedure using the endoscope 18 on the subject 20.

The tablet terminal 32 is provided with a touch panel display 44. The touch panel display 44 has a display (liquid crystal display or EL display, for example) and a touch panel. For example, the touch panel display 44 is formed by overlaying the touch panel on the display. One example of the touch panel display 44 is an out-cell touch panel display in which the touch panel is overlaid on the surface of the display area of the display. Note that this is merely one example, and the touch panel display 44 may also be an on-cell or in-cell touch panel display, for example.

Various screens are displayed on the touch panel display 44. The various screens displayed on the touch panel display 44 and the screens 36 and 38 displayed on the display apparatus 30 are viewed by the user in a manner allowing for visual comparison.

One example of a screen displayed on the touch panel display 44 is a past examination image screen 46. On the past examination image screen 46, multiple examination result images 50 are displayed in a list, and subject-identifying information 52 is displayed adjacently to the multiple examination result images 50. Each of the multiple examination result images 50 is an endoscopic image 40 obtained by imaging multiple locations (multiple locations on the inner wall of the large intestine, for example) inside the body of the subject 20 with the endoscope 18 in the first endoscopic examination of the subject 20. The subject-identifying information 52 is the same information as the information included in the subject-identifying information 42.

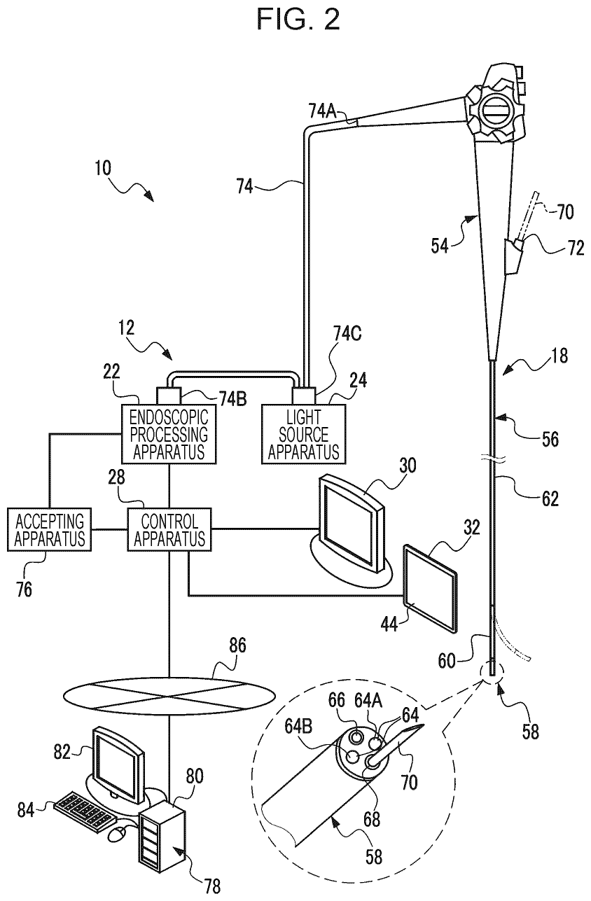

As illustrated by way of example in FIG. 2, the endoscope 18 is provided with a manipulation part 54 and an insertion part 56. The insertion part 56 is formed into a tubular shape. The insertion part 56 has a leading end part 58, a curving part 60, and a flexible part 62. The leading end part 58, curving part 60, and flexible part 62 are disposed from the leading-end side to the base-end side of the insertion part 56 in the order of the leading end part 58, curving part 60, and the flexible part 62. The flexible part 62 is formed from a long, flexible material, and connects the manipulation part 54 with the curving part 60. Manipulating the manipulation part 54 causes the curving part 60 to partially curve or rotate about the axis of the insertion part 56. As a result, the insertion part 56 is fed deeper into a luminal organ while curving or rotating about the axis of the insertion part 56 according to the shape of a luminal organ (the shape of the large intestinal tract, for example).

An illumination apparatus 64, a camera 66, and a treatment tool aperture 68 are provided in the leading end part 58. The illumination apparatus 64 has an illumination window 64A and an illumination window 64B. The illumination apparatus 64 radiates light through the illumination window 64A and the illumination window 64B. The type of light radiated from the illumination apparatus 64 may be visible light (white light, for example), non-visible light (near-infrared light, for example), and/or special light, for example. The special light may be light for BLI and/or light for LCI, for example. The camera 66 images the inside of a luminal organ using an optical method. One example of the camera 66 is a CMOS camera. A CMOS camera is merely one example, and the camera 66 may also be another type of camera, such as a CCD camera.

The treatment tool aperture 68 is an aperture for allowing a treatment tool 70 to protrude from the leading end part 58. The treatment tool aperture 68 also functions as an aspiration port to aspirate blood, internal contaminants, and the like. A treatment tool insertion port 72 is formed in the manipulation part 54, and the treatment tool 70 is inserted into the insertion part 56 from the treatment tool insertion port 72. The treatment tool 70 passes through the interior of the insertion part 56 to protrude out from the treatment tool aperture 68. In the example illustrated in FIG. 2, a puncture needle is illustrated as the treatment tool 70. Other examples of the treatment tool 70 include a wire, a scalpel, grasping forceps, a guide sheath, and an ultrasound probe.

The endoscope apparatus 12 is provided with a universal cord 74 and an accepting apparatus 76.

The universal cord 74 has a base end part 74A, a first leading end part 74B, and a second leading end part 74C. The base end part 74A is connected to the manipulation part 54. The first leading end part 74B is connected to the endoscopic processing apparatus 22. The second leading end part 74C is connected to the light source apparatus 24.

The accepting apparatus 76 accepts an instruction from the user and outputs the accepted instruction as an electrical signal. Examples of the accepting apparatus 76 include a footswitch, a keyboard, a mouse, a touch panel, and a microphone.

The accepting apparatus 76 is connected to the endoscopic processing apparatus 22. The endoscopic processing apparatus 22 exchanges various signals with the camera 66 according to instructions accepted by the accepting apparatus 76 and controls the light source apparatus 24. The endoscopic processing apparatus 22 causes the camera 66 to perform imaging, and acquires and outputs the endoscopic image 40 (see FIG. 1) from the camera 66. The light source apparatus 24 emits light under control by the endoscopic processing apparatus 22, and supplies the light to the illumination apparatus 64. A light guide is built into the illumination apparatus 64, and light supplied from the light source apparatus 24 is radiated from the illumination windows 64A and 64B via the light guide.

The control apparatus 28 controls the endoscope apparatus 12 overall. The endoscopic processing apparatus 22, the display apparatus 30, the tablet terminal 32, and the accepting apparatus 76 are connected to the control apparatus 28. The control apparatus 28 controls the display apparatus 30 and the tablet terminal 32 according to instructions accepted by the accepting apparatus 76. The control apparatus 28 acquires the endoscopic image 40 from the endoscopic processing apparatus 22 and causes the display apparatus 30 to display the screen 36 including the acquired endoscopic image 40, or causes the display apparatus 30 to display the screen 38 including the subject-identifying information 42 (see FIG. 1). The control apparatus 28 also causes the touch panel display 44 of the tablet terminal 32 to selectively display multiple screens, including the past examination image screen 46 (see FIG. 1). The control apparatus 28 is one example of an “information processing apparatus” according to the technology of the present disclosure.

The endoscope system 10 is provided with a server 78. The server 78 includes a computer 80 which is the main part of the server 78, a display apparatus 82, and an accepting apparatus 84. The computer 80 and the control apparatus 28 are communicatively connected over a network 86. One example of the network 86 is a LAN. Note that a LAN is merely one example, and the network 86 may be formed using at least one of a LAN, a WAN, or the like.

The control apparatus 28 is positioned as a client terminal to the server 78. Consequently, the server 78 executes processing in response to a request given from the control apparatus 28 over the network 86, and provides a processing result to the control apparatus 28 over the network 86.

The display apparatus 82 and the accepting apparatus 84 are connected to the computer 80. The display apparatus 82 displays various information under control by the computer 80. The display apparatus 82 may be a liquid crystal display or an EL display, for example. The accepting apparatus 84 accepts instructions from a user or the like of the server 78. The accepting apparatus 84 may be a keyboard and mouse, for example. The computer 80 executes processing according to instructions accepted by the accepting apparatus 84.

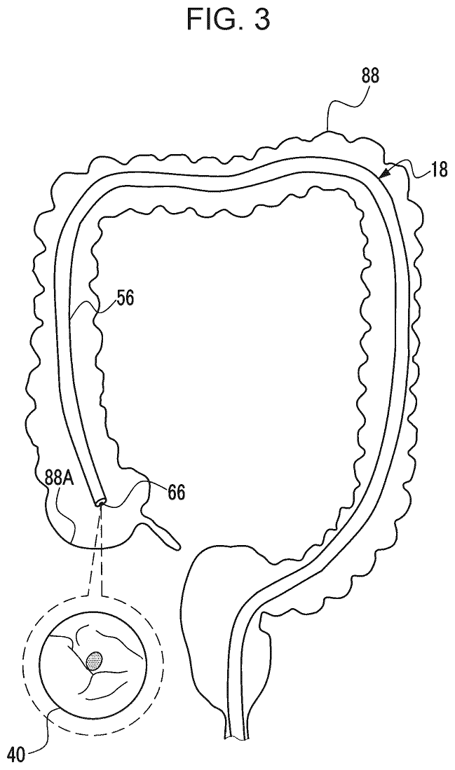

As illustrated by way of example in FIG. 3, the insertion part 56 of the endoscope 18 is inserted into the large intestine 88 from the anus of the subject 20. The camera 66 generates the endoscopic image 40 by imaging the interior of the large intestine 88. The endoscopic image 40 is generated as an image illustrating the state of the inner wall 88A. For example, the camera 66 inserted into the large intestine 88 proceeds deeper from the entrance of the large intestine 88 and images the inner wall 88A according to a predetermined frame rate as the camera 66 proceeds deeper from the entrance of the large intestine 88. This generates a dynamic image including multiple frames of endoscopic images 40 illustrating the state of the inner wall 88A as the camera 66 proceeds deeper from the entrance of the large intestine 88.

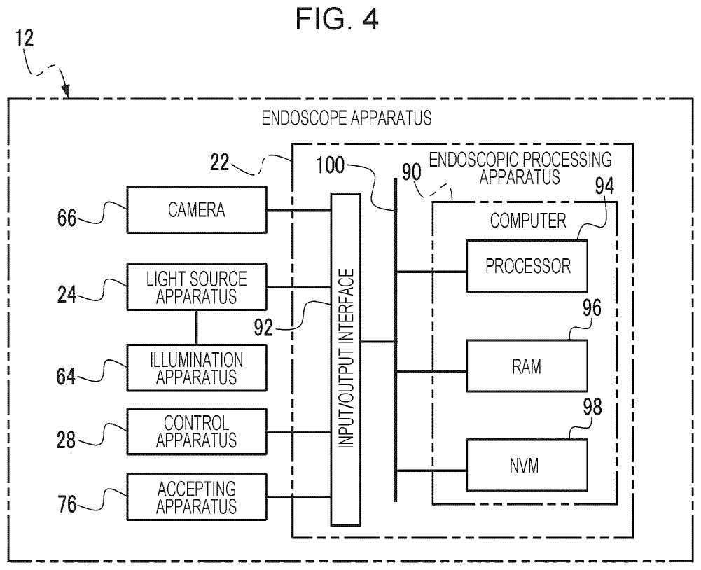

As illustrated by way of example in FIG. 4, the endoscopic processing apparatus 22 is provided with a computer 90 and an input/output interface 92. The computer 90 is provided with a processor 94, RAM 96, and NVM 98. The input/output interface 92, processor 94, RAM 96, and NVM 98 are connected to a bus 100.

The processor 94 includes a CPU and GPU, for example, and controls the endoscopic processing apparatus 22 overall. The GPU operates under control by the CPU and is mainly responsible for executing image processing. Note that the processor 94 may also be one or more CPUs with integrated GPU functionality, or one or more CPUs without integrated GPU functionality.

The RAM 96 is a memory in which information is stored temporarily, and is used as work memory by the processor 94. The NVM 98 is a non-volatile storage apparatus storing various programs, various parameters, and the like. The NVM 98 may be flash memory (EEPROM, for example) and/or an SSD, for example. Note that flash memory and an SSD are merely one example, and the NVM 98 may also be another type of non-volatile storage apparatus, such as an HDD, and may also be a combination of two or more types of non-volatile storage apparatuses.

The accepting apparatus 76 is connected to the input/output interface 92, and the processor 94 acquires an instruction accepted by the accepting apparatus 76 via the input/output interface 92 and executes processing according to the acquired instruction. Also, the camera 66 is connected to the input/output interface 92. The processor 94 controls the camera 66 via the input/output interface 92 and acquires, via the input/output interface 92, the endoscopic image 40 obtained by having the camera 66 image the inside of the body the subject 20. Also, the light source apparatus 24 is connected to the input/output interface 92. The processor 94 controls the light source apparatus 24 via the input/output interface 92 to thereby supply light to the illumination apparatus 64 and regulate the amount of light supplied to the illumination apparatus 64. Also, the control apparatus 28 is connected to the input/output interface 92. The processor 94 exchanges various signals with the control apparatus 28 via the input/output interface 92.

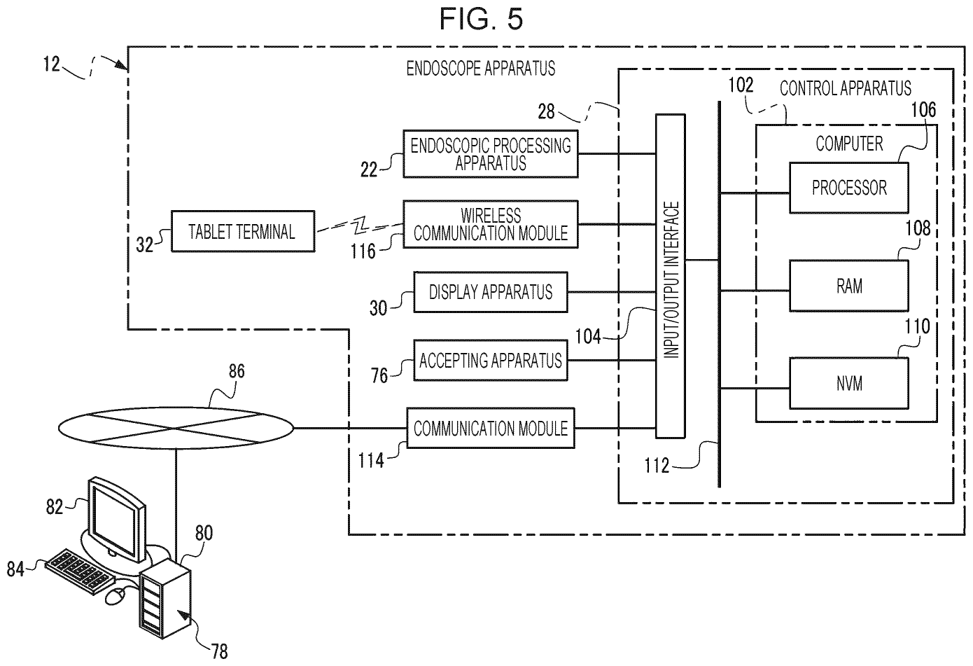

As illustrated by way of example in FIG. 5, the control apparatus 28 is provided with a computer 102 and an input/output interface 104. The computer 102 is provided with a processor 106, RAM 108, and NVM 110. The input/output interface 104, processor 106, RAM 108, and NVM 110 are connected to a bus 112.

The processor 106 controls the control apparatus 28 overall. Note that the multiple hardware resources (that is, the processor 106, RAM 108, and NVM 110) included in the computer 102 illustrated in FIG. 5 are of a similar type to the multiple hardware resources included in the computer 90 illustrated in FIG. 4, and therefore a description is omitted here.

The accepting apparatus 76 is connected to the input/output interface 104, and the processor 106 acquires an instruction accepted by the accepting apparatus 76 via the input/output interface 104 and executes processing according to the acquired instruction. Also, the endoscopic processing apparatus 22 is connected to the input/output interface 104, and the processor 106 exchanges various signals with the processor 94 of the endoscopic processing apparatus 22 (see FIG. 4) via the input/output interface 104.

The display apparatus 30 is connected to the input/output interface 104, and the processor 106 controls the display apparatus 30 via the input/output interface 104, thereby causing the display apparatus 30 to display various information. For example, the processor 106 acquires the endoscopic image 40 (see FIG. 1) from the endoscopic processing apparatus 22 and causes the display apparatus 30 to display the endoscopic image 40.

The endoscope apparatus 12 is provided with a communication module 114. The communication module 114 is connected to the input/output interface 104. The communication module 114 is an interface including a communication processor, an antenna, and the like. The communication module 114 is connected to the network 86 and directs communication between the processor 106 and the computer 80 of the server 78.

The endoscope apparatus 12 is provided with a wireless communication module 116. The wireless communication module 116 is connected to the input/output interface 104. The wireless communication module 116 is an interface including a wireless communication processor, an antenna, and the like. The wireless communication module 116 is communicatively connected to the tablet terminal 32 over a wireless LAN or the like, and directs communication between the processor 106 and the tablet terminal 32. Note that although the description herein gives an example in which the control apparatus 28 and the tablet terminal 32 communicate in a wireless manner, this is merely one example, and the control apparatus 28 and the tablet terminal 32 may also communicate in a wired manner.

Note that the computer 102 is an example of a “computer” according to the technology of the present disclosure. Also, the processor 106 is an example of a “processor” according to the technology of the present disclosure. Also, the display apparatus 30 and the tablet terminal 32 are each an example of a “display apparatus” according to the technology of the present disclosure.

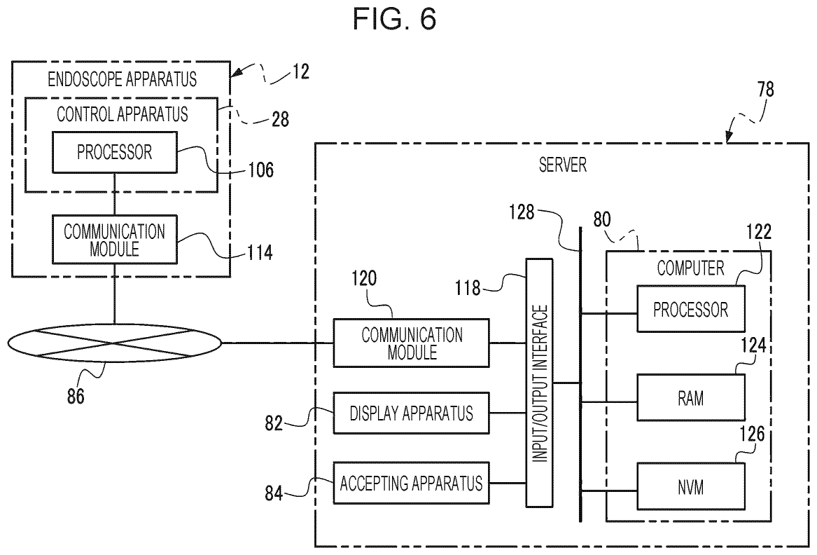

As illustrated by way of example in FIG. 6, the server 78 is provided with an input/output interface 118 similar to the input/output interface 104 (see FIG. 5) and a communication module 120 similar to the communication module 114 (see FIG. 5), in addition to the display apparatus 82 and the accepting apparatus 84. The computer 80 is provided with a processor 122 similar to the processor 106 (see FIG. 5), RAM 124 similar to the RAM 108 (see FIG. 5), and NVM 126 similar to the NVM 110 (see FIG. 5). The input/output interface 118, processor 122, RAM 124, and NVM 126 are connected to a bus 128.

The display apparatus 82 is connected to the input/output interface 118, and the processor 122 controls the display apparatus 82 via the input/output interface 118, thereby causing the display apparatus 82 to display various information.

The accepting apparatus 84 is connected to the input/output interface 118, and the processor 122 acquires an instruction accepted by the accepting apparatus 84 via the input/output interface 118 and executes processing according to the acquired instruction.

The communication module 120 is connected to the input/output interface 118. The communication module 120 is connected to the network 86 and directs communication between the processor 122 of the server 78 and the processor 106 of the control apparatus 28 by cooperating with the communication module 114.

Incidentally, multiple endoscopic images 40 obtained by imaging with the endoscope 18 in the first endoscopic examination of the subject 20 using the endoscope 18 are recorded in a report or the like. The physician 14 identifies a location (a lesion, for example) to be treated (cured, for example) inside the body of the subject 20 in the second endoscopic examination while looking the multiple endoscopic images 40 recorded in a report or the like.

To perform the second endoscopic examination on the subject 20, it is important for all of the physician 14 and auxiliary staff member 16 to understand the extent to which the location (hereinafter referred to as the “main examination target site”) to be treated inside the body of the subject 20 in the second endoscopic examination has changed since the time the first endoscopic examination was performed. One conceivable method of realizing this is to have all of the physician 14 and auxiliary staff member 16 understand the main examination target site by using the period of carrying out a timeout (that is, a discussion held before or during the second endoscopic examination), for example. In this case, it is preferable to select an appropriate endoscopic image 40 (that is, an endoscopic image 40 showing the main examination target site) from among the multiple endoscopic images 40 recorded in a report or the like, and present the selected endoscopic image 40 to all of the physician 14 and auxiliary staff member 16.

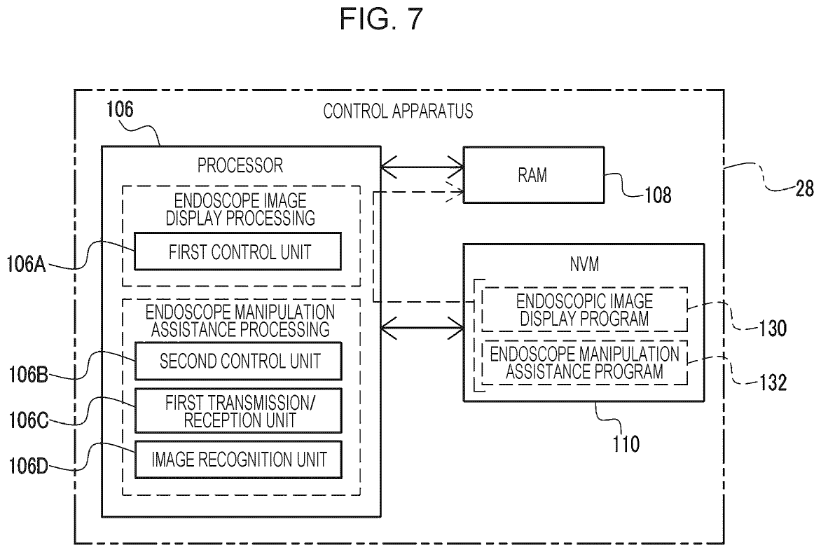

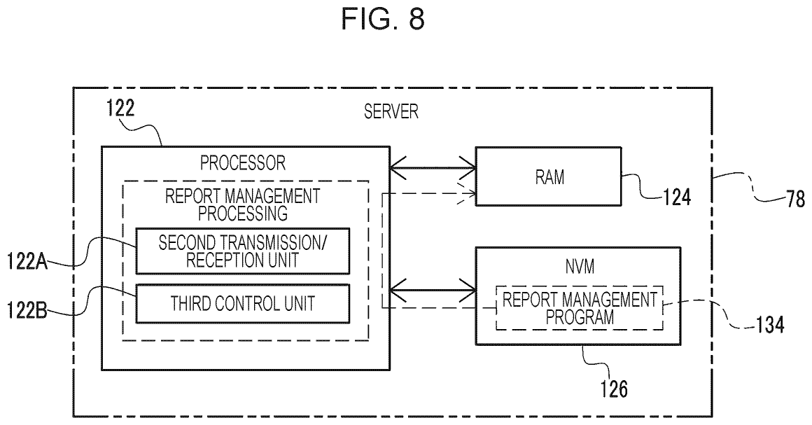

Accordingly, in view of such circumstances, in the present embodiment, endoscopic image display processing and endoscope manipulation assistance processing are performed by the processor 106 of the control apparatus 28 (see FIG. 7), and report management processing is performed by the processor 122 of the server 78 (see FIG. 8).

As illustrated by way of example in FIG. 7, an endoscopic image display program 130 is stored in the NVM 110. The processor 106 reads out the endoscopic image display program 130 from the NVM 110 and executes the read-out endoscopic image display program 130 in the RAM 108. The endoscopic image display processing is realized by the processor 106 operating as a first control unit 106A in accordance with the endoscopic image display program 130 executed in the RAM 108.

An endoscope manipulation assistance program 132 is stored in the NVM 110. The processor 106 reads out the endoscope manipulation assistance program 132 from the NVM 110 and executes the read-out endoscope manipulation assistance program 132 in the RAM 108. The endoscope manipulation assistance processing is realized by the processor 106 operating as a second control unit 106B, a first transmission/reception unit 106C, and an image recognition unit 106D in accordance with the endoscope manipulation assistance program 132 executed in the RAM 108. The endoscope manipulation assistance program 132 is an example of a “program” according to the technology of the present disclosure.

As illustrated by way of example in FIG. 8, a report management program 134 is stored in the NVM 126. The processor 122 reads out the report management program 134 from the NVM 126 and executes the read-out report management program 134 in the RAM 124. The report management processing is realized by the processor 122 operating as a second transmission/reception unit 122A and a third control unit 122B in accordance with the report management program 134 executed in the RAM 124.

Note that in the following, the endoscopic image display processing and the endoscope manipulation assistance processing are collectively referred to as the “control apparatus processing” in some cases. Also, in the following, the endoscopic image display processing, the endoscope manipulation assistance processing, and the report management processing are collectively referred to as the “endoscope system processing” in some cases out of convenience. Also, in the following, the endoscopic image display program 130, the endoscope manipulation assistance program 132, and the report management program 134 are collectively referred to as the “endoscope system program” in some cases out of convenience.

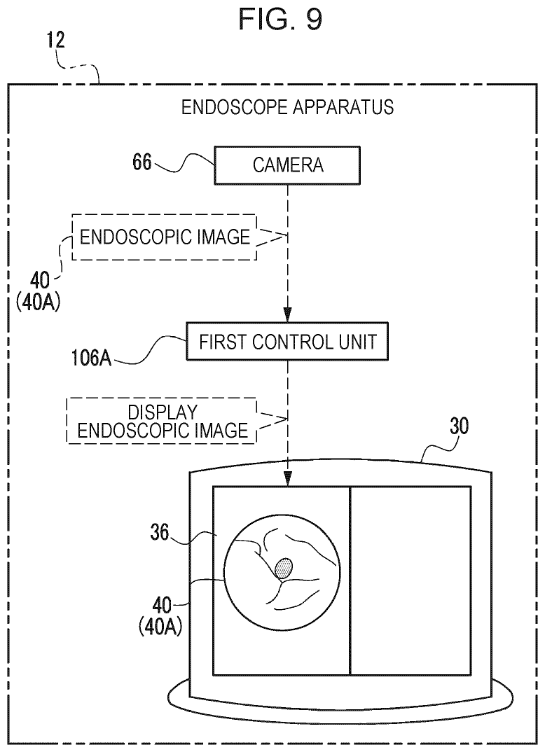

As illustrated by way of example in FIG. 9, in the endoscope apparatus 12, the first control unit 106A causes the display apparatus 30 to display the screen 36. The first control unit 106A acquires endoscopic images 40 from the camera 66 according to a predetermined frame rate, and displays the acquired endoscopic images 40 sequentially on the screen 36. This causes a dynamic image formed from the multiple endoscopic images 40 to be displayed on the screen 36. The endoscopic images 40 displayed on the screen 36 are images obtained in the second endoscopic examination. Note that in the following, the endoscopic image 40 obtained in the second endoscopic examination is referred to as the “endoscopic image 40A” when it is necessary to distinguish the endoscopic image 40 obtained during the second endoscopic examination from the endoscopic image 40 obtained in the first endoscopic examination. The endoscopic image 40A is an example of a “second endoscopic examination image” according to the technology of the present disclosure.

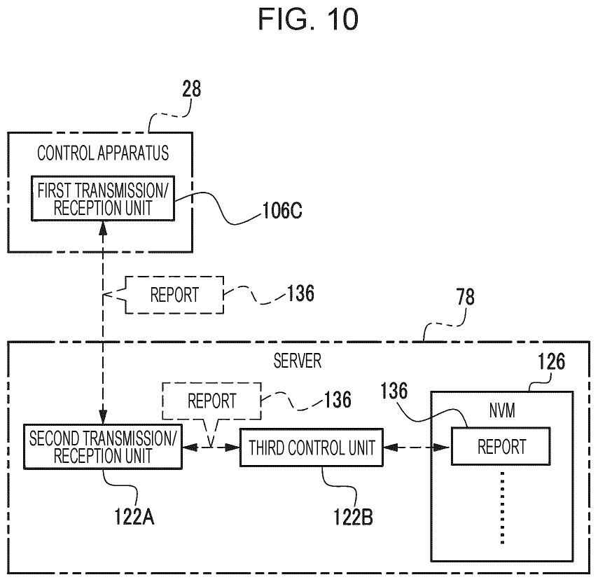

As illustrated by way of example in FIG. 10, multiple reports 136 are archived in the NVM 126 of the server 78. For example, a different report 136 is created for each subject 20. The report 136 contains information pertaining to the subject 20 and information pertaining to the results of the first endoscopic examination performed on the subject 20. The third control unit 122B stores the report 136 in the NVM 126 and acquires the report 136 from the NVM 126. The second transmission/reception unit 122A transmits the report 136 acquired from the NVM 126 by the third control unit 122B to the control apparatus 28. The report 136 transmitted by the second transmission/reception unit 122A is received by the first transmission/reception unit 106C of the control apparatus 28. Also, the first transmission/reception unit 106C of the control apparatus 28 transmits the report 136 to the server 78. The report 136 transmitted by the first transmission/reception unit 106C is received by the second transmission/reception unit 122A. The report 136 received by the second transmission/reception unit 122A is stored in the NVM 126 by the third control unit 122B. The NVM 126 is an example of “storage” and an “apparatus” according to the technology of the present disclosure.

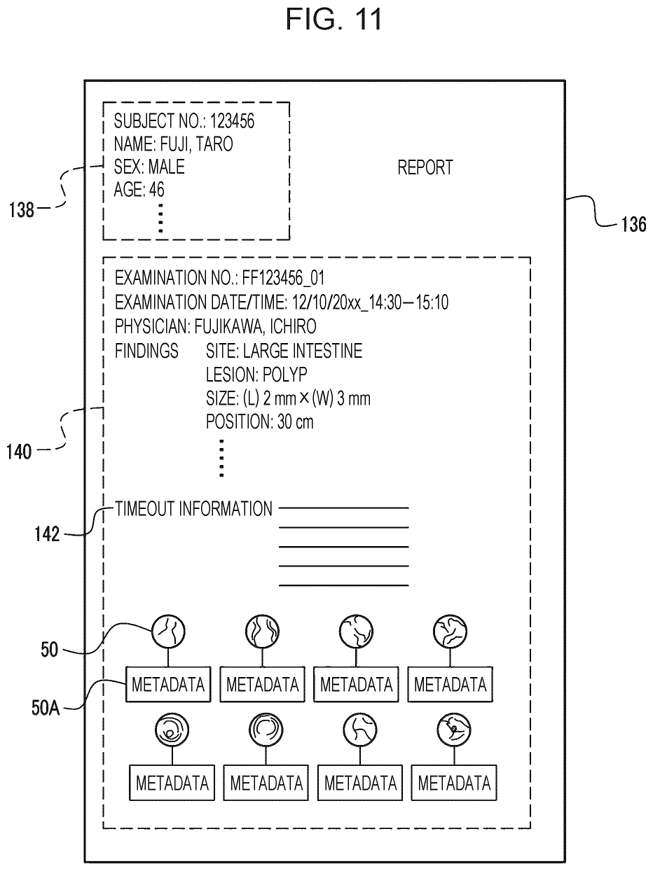

As illustrated by way of example in FIG. 11, subject-identifying information 138 and endoscopic examination information 140 are recorded in the report 136. The subject-identifying information 138 is information that can be used to uniquely identify the subject 20. The subject-identifying information 138 includes a subject number, which is a number uniquely assigned to the subject 20, the name of the subject 20, the sex of the subject 20, the age of the subject 20, and the like. The endoscopic examination information 140 is information pertaining to the first endoscopic examination performed on the subject 20 identified from the subject-identifying information 138. The endoscopic examination information 140 includes an examination number, which is a number uniquely assigned to the first endoscopic examination, an examination date and time indicating when the first endoscopic examination was performed, the name of the physician who performed the first endoscopic examination, and findings (such as the site on which the first endoscopic examination was performed and the type, size, and location of a lesion discovered by the first endoscopic examination).

The endoscopic examination information 140 also includes timeout information 142 and multiple examination result images 50. The timeout information 142 is information obtained in a timeout that was performed during the period of carrying out the first endoscopic examination identified from the examination number recorded in the report 136. The information obtained in a timeout includes information (such as names or identification numbers) that can be used to identify the persons who participated in the timeout, the matters confirmed during the timeout, the date and time when the timeout was performed, information indicating the place where the timeout was performed, and the like.

The examination result image 50 is the endoscopic image 40 obtained in the first endoscopic examination identified from the examination number recorded in the report 136. Metadata 50A is associated with the examination result image 50 as data that accompanies the examination result image 50. The metadata 50A includes, for example, various information pertaining to the examination result image 50 (such as the date and time when the examination result image 50 was obtained and the result of performing image recognition processing on the examination result image 50) and the same information as the timeout information 142.

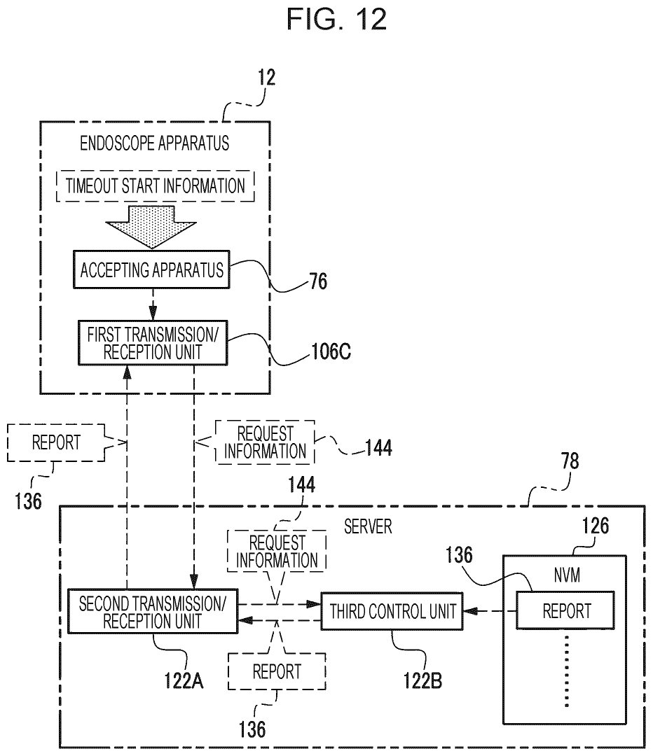

As illustrated by way of example in FIG. 12, when a timeout is started, the accepting apparatus 76 accepts timeout start information. The timeout start information is information indicating that a timeout has started. Also, when the timeout ends, the accepting apparatus 76 accepts timeout end information. The timeout end information is information indicating that a timeout has ended. The accepting apparatus 76 accepts the timeout start information from the user at the timing when the timeout is started, and accepts the timeout end information at the timing when the timeout is ended. When the timeout start information is accepted by the accepting apparatus 76, the processor 106 recognizes that the timeout has started. Also, when the timeout end information is accepted by the accepting apparatus 76, the processor 106 recognizes that the timeout has ended.

When the timeout start information is accepted by the accepting apparatus 76, the first transmission/reception unit 106C transmits to the server 78 request information 144, which is information requesting the server 78 to transmit the report 136. The request information 144 includes information that can be used to uniquely identify the report 136. The information that can be used to uniquely identify the report 136 may be the subject number and/or the examination number.

The request information 144 transmitted by the first transmission/reception unit 106C is received by the second transmission/reception unit 122A of the server 78. When the request information 144 is received by the second transmission/reception unit 122A, the third control unit 122B acquires, from the NVM 126, the report 136 corresponding to the request information 144 (for example, the report 136 identified from the subject number and/or the examination number). The second transmission/reception unit 122A transmits the report 136 acquired from the NVM 126 by the third control unit 122B to the endoscope apparatus 12. The report 136 transmitted by the second transmission/reception unit 122A is received by the first transmission/reception unit 106C of the endoscope apparatus 12. Note that the reception of the report 136 by the first transmission/reception unit 106C is an example of the “acquisition of multiple first endoscopic examination images” according to the technology of the present disclosure.

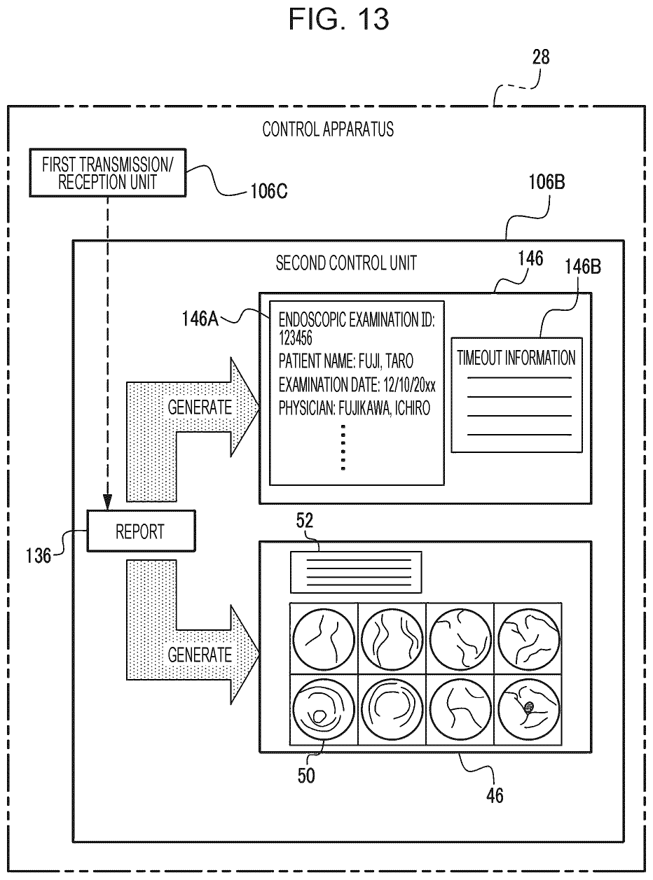

As illustrated by way of example in FIG. 13, in the control apparatus 28, the second control unit 106B generates the past examination image screen 46 and a subject information screen 146 on the basis of the report 136 received by the first transmission/reception unit 106C. The past examination image screen 46 contains the multiple examination result images 50 and the subject-identifying information 52.

The multiple examination result images 50 on the past examination image screen 46 are multiple examination result images 50 recorded in the report 136 received by the first transmission/reception unit 106C as multiple endoscopic images 40 obtained for multiple sites (multiple locations inside the large intestine 88, for example) in the first endoscopic examination (see FIG. 11). Metadata 50A is also associated with the examination result images 50 on the past examination image screen 46. The subject-identifying information 52 is information (such as the subject number and the name, sex, and age of the subject) included among the subject-identifying information 138 recorded in the report 136 received by the first transmission/reception unit 106C. The subject-identifying information 52 is an example of “second information” according to the technology of the present disclosure and is used as information required in a timeout performed during the period of carrying out the second endoscopic examination.

The subject information screen 146 includes subject-identifying information 146A and timeout information 146B. The subject-identifying information 146A is information that can be used to uniquely identify the subject 20. The subject-identifying information 146A is an example of “second information” according to the technology of the present disclosure and is used as information required in a timeout performed during the period of carrying out the second endoscopic examination. The subject-identifying information 146A may be the same information as the subject-identifying information 138 included in the report 136 received by the first transmission/reception unit 106C, for example. The timeout information 146B is information obtained in a timeout that was performed during the period of carrying out the first endoscopic examination identified from the examination number recorded in the report 136 received by the first transmission/reception unit 106C. The timeout information 146B may be the same information as the timeout information 142 included in the report 136 received by the first transmission/reception unit 106C, for example. The timeout information 146B is an example of “first information” according to the technology of the present disclosure.

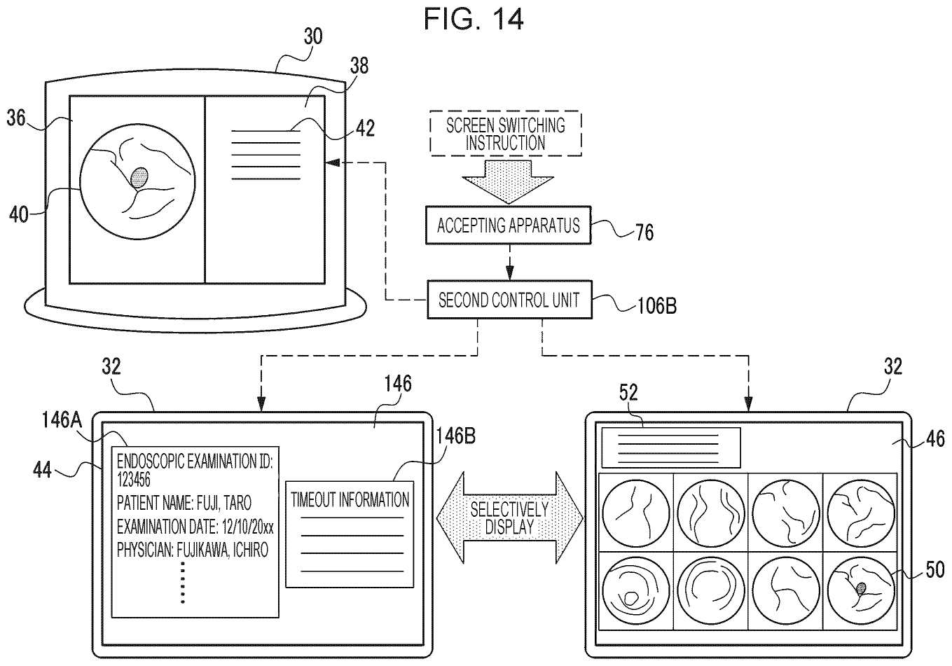

As illustrated by way of example in FIG. 14, the second control unit 106B causes the display apparatus 30 to display the screen 38 beside the screen 36. The second control unit 106B acquires the subject-identifying information 138 from the report 136 illustrated in FIG. 13, and displays the acquired subject-identifying information 138 as the subject-identifying information 42 on the screen 38.

The second control unit 106B also causes the touch panel display 44 of the tablet terminal 32 to display the past examination image screen 46 or the subject information screen 146. The accepting apparatus 76 accepts a screen switching instruction, which is an instruction to switch the display between the past examination image screen 46 and the subject information screen 146. In response to the screen switching instruction accepted by the accepting apparatus 76, the second control unit 106B causes the touch panel display 44 to selectively display the past examination image screen 46 and the subject information screen 146.

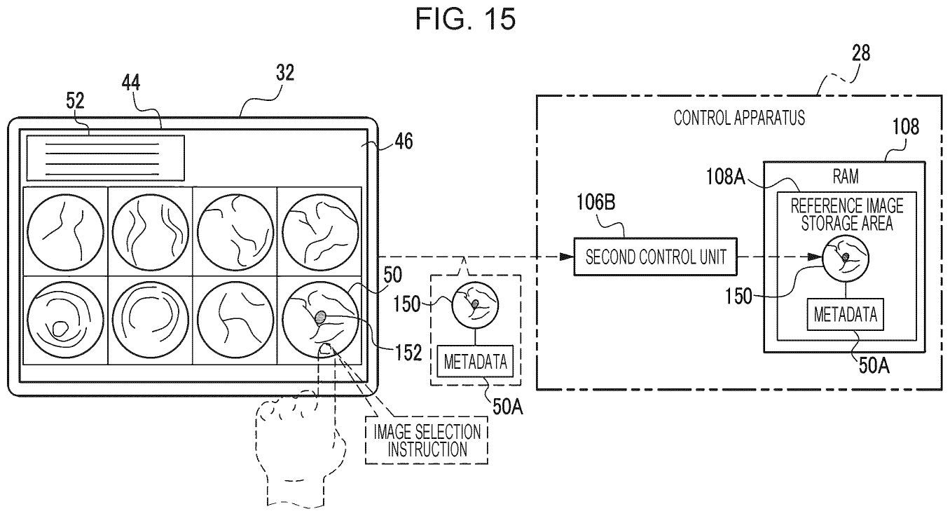

As illustrated by way of example in FIG. 15, while the past examination image screen 46 is being displayed on the touch panel display 44, the user gives to the touch panel display 44 an image selection instruction, which is an instruction to select one of the examination result images 50 as a reference image 150. The reference image 150 refers an image that is referred to by the user. The example illustrated in FIG. 15 illustrates a situation in which an examination result image 50 showing a characteristic portion 152, which is a portion indicating a lesion identified from the endoscopic examination information 140 (see FIG. 11) in the report 136, is selected as the reference image 150. The characteristic portion 152 is an example of a “characteristic portion within a reference image” and a “first characteristic portion” according to the technology of the present disclosure. The image selection instruction is an example of an “instruction accepted by the accepting apparatus” according to the technology of the present disclosure.

On the tablet terminal 32, one examination result image 50 is selected as the reference image 150 according to the image selection instruction. In the control apparatus 28, the second control unit 106B acquires, from the tablet terminal 32, the reference image 150 selected by the image selection instruction and the metadata 50A associated with the examination result image 50 selected as the reference image 150. The second control unit 106B stores the reference image 150 and the metadata 50A acquired from the tablet terminal 32 in an associated state in a reference image storage area 108A in the RAM 108.

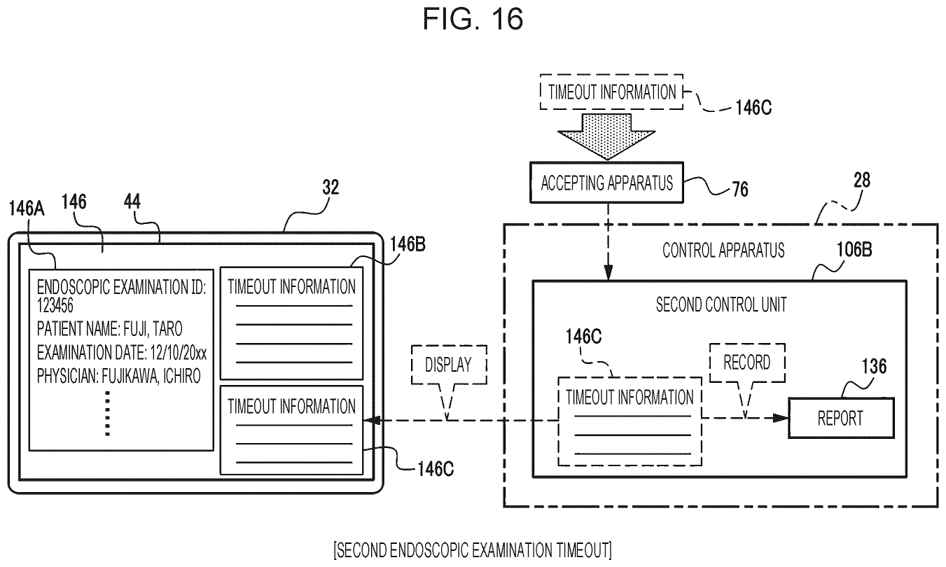

As illustrated by way of example in FIG. 16, timeout information 146C, which is information obtained in a timeout of the second endoscopic examination, is accepted by the accepting apparatus 76. Additionally, in the control apparatus 28, the second control unit 106B records the timeout information 146C accepted by the accepting apparatus 76 in the report 136. The “recording” may be appending, for example. Recording the timeout information 146C in the report 136 in this way updates the content of the report 136. Also, the second control unit 106B displays the timeout information 146C accepted by the accepting apparatus 76 on the subject information screen 146 of the touch panel display 44. On the subject information screen 146, the timeout information 146C is displayed adjacently to the subject-identifying information 146A and the timeout information 146B.

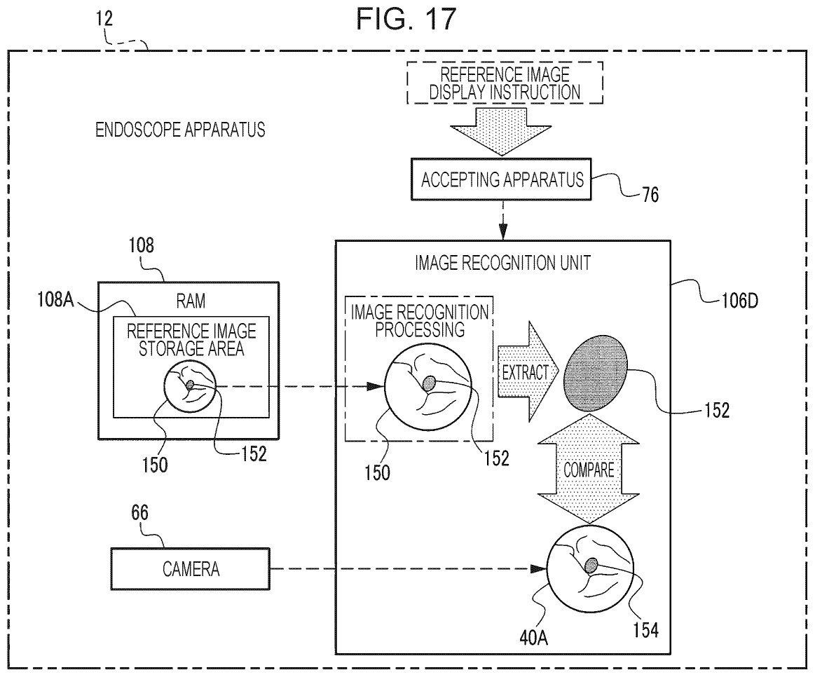

As illustrated by way of example in FIG. 17, in the endoscope apparatus 12, the accepting apparatus 76 accepts a reference image display instruction. The reference image display instruction is an instruction causing the touch panel display 44 to display the reference image 150. The reference image display instruction is given by the user during the period of carrying out the timeout of the second endoscopic examination. When the reference image display instruction is accepted by the accepting apparatus 76, the image recognition unit 106D acquires the reference image 150 from the reference image storage area 108A. The image recognition unit 106D extracts the characteristic portion 152 from the reference image 150 by performing image recognition processing based on AI, template matching, or other approach (hereinafter simply referred to as “image recognition processing”) on the reference image 150. The image recognition unit 106D acquires the endoscopic image 40A from the camera 66. The image recognition unit 106D then compares the endoscopic image 40A acquired from the camera 66 with the characteristic portion 152 extracted from the reference image 150. By comparing the endoscopic image 40A with the characteristic portion 152, the image recognition unit 106D determines whether or not the endoscopic image 40A shows a corresponding characteristic portion 154. The corresponding characteristic portion 154 is an example of a “location corresponding to a characteristic portion” and a “second characteristic portion” according to the technology of the present disclosure.

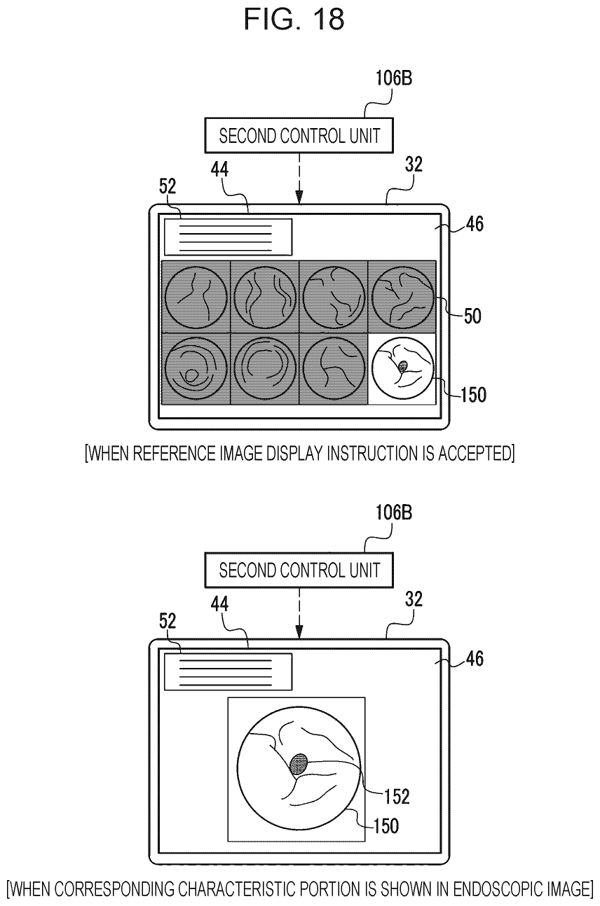

On the other hand, when the reference image display instruction is accepted by the accepting apparatus 76, the examination result image 50 selected according to the image selection instruction illustrated in FIG. 15 is confirmed as the reference image 150. That is, when the reference image display instruction is accepted by the accepting apparatus 76, the examination result image 50, which is an image satisfying a specific condition (as an example in this case, the image selected according to the image selection instruction), is confirmed as the reference image 150. When the reference image display instruction is accepted by the accepting apparatus 76, as illustrated by way of example in FIG. 18, the second control unit 106B displays the reference image 150 in a manner distinguishable from the other examination result images 50 on the past examination image screen 46. The reference image 150 is displayed on the past examination image screen 46 in the timeout phase performed during the period of carrying out the second endoscopic examination. For example, the second control unit 106B displays the reference image 150 in a distinguishable manner by displaying the examination result images 50 other than the reference image 150 in a grayed-out manner (see the upper illustration in FIG. 18) or in a semi-transparent manner, or by not displaying the examination result images 50 other than the reference image 150.

Note that the condition stipulating that an image selection instruction has been accepted and a reference image display instruction has been accepted by the accepting apparatus 76 is an example of a “specific condition” and a “condition stipulating that a selection has been made according to an instruction accepted by an accepting apparatus” according to the technology of the present disclosure.

As illustrated by way of example in FIG. 18, when the image recognition unit 106D has determined that the endoscopic image 40A shows the corresponding characteristic portion 154, the second control unit 106B performs processing to provide a notification that the corresponding characteristic portion 154 is shown in the endoscopic image 40A by displaying the reference image 150 in an enlarged manner on the past examination image screen 46. The processing to provide a notification that the corresponding characteristic portion 154 is shown in the endoscopic image 40A is an example of “first notification processing” according to the technology of the present disclosure. Although an example form of displaying the reference image 150 in an enlarged manner is given herein, this form is merely one example. For example, the notification that the corresponding characteristic portion 154 is shown in the endoscopic image 40A may also be provided by highlighting the outline of the reference image 150 or by displaying a message, mark, or the like that can be used to specify that the corresponding characteristic portion 154 is shown in the endoscopic image 40A.

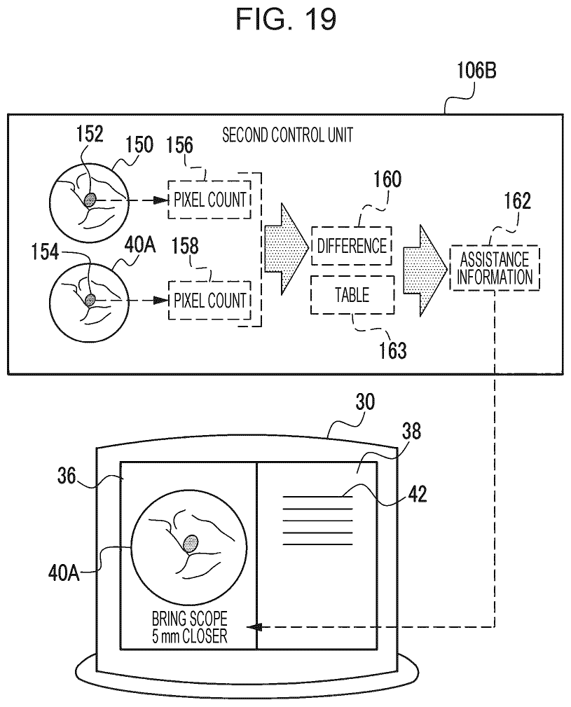

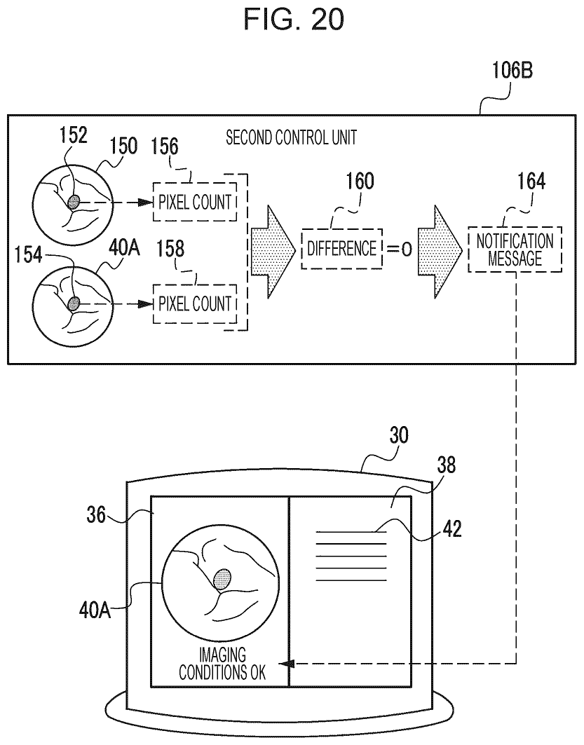

The following describes assistance processing for assisting with making the imaging conditions of the second endoscopic examination consistent with the imaging conditions of the first endoscopic examination. As an example, in the case where the scale (that is, the angle of view) of the reference image 150 and the scale of the endoscopic image 40A are different from each another, the assistance processing may be processing to make the scale of the endoscopic image 40A consistent with the scale of the reference image 150. Accordingly, the following describes processing to make the scale of the endoscopic image 40A consistent with the scale of the reference image 150, with reference to FIGS. 19 and 20.

A first assumption and a second assumption are conceivable assumptions that could be made when making the scale of the endoscopic image 40A consistent with the scale of the reference image 150. The first assumption is that the actual size of the characteristic portion 152 shown in the reference image 150 and the actual size of the corresponding characteristic portion 154 shown in the endoscopic image 40A are matching. The second assumption is that the actual size of the characteristic portion 152 shown in the reference image 150 and the actual size of the corresponding characteristic portion 154 shown in the endoscopic image 40A are not matching.

Under the first assumption, as illustrated by way of example in FIG. 19, the second control unit 106B acquires a pixel count 156 of the characteristic portion 152 from the reference image 150 and acquires a pixel count 158 of the corresponding characteristic portion 154 from the endoscopic image 40A. The second control unit 106B calculates the difference 160 between the pixel count 156 and the pixel count 158. The second control unit 106B derives assistance information 162 according to the difference 160. The assistance information 162 is derived using a table 163 in which the difference 160 and the assistance information 162 are associated with each other. The assistance information 162 is information required to make the scale of the endoscopic image 40A consistent with the scale of the reference image 150. The information required to make the scale of the endoscopic image 40A consistent with the scale of the reference image 150 refers to a message guiding the position of the camera 66 to a position where the difference 160 is zero, for example. The second control unit 106B outputs the assistance information 162 according to the difference 160 to the display apparatus 30. This causes the assistance information 162 to be displayed on the screen 36 of the display apparatus 30. The assistance information 162 is an example of “assistance information” according to the technology of the present disclosure. The processing whereby the second control unit 106B outputs the assistance information 162 to the display apparatus 30 is an example of “output processing” according to the technology of the present disclosure.

As illustrated by way of example in FIG. 20, every time a new endoscopic image 40A is obtained by the camera 66, the second control unit 106B calculates the difference 160 in a manner similar to the above, and determines whether or not the difference 160 is zero. If the difference 160 is zero, the second control unit 106B performs processing to provide a notification that the scale of the reference image 150 and the scale of the endoscopic image 40A are matching by generating a notification message 164 and displaying the generated notification message 164 on the screen 36. The processing to provide a notification that the scale of the reference image 150 and the scale of the endoscopic image 40A are matching is an example of “assistance processing” and “third notification processing” according to the technology of the present disclosure. The notification message 164 is a message notifying that the scale of the reference image 150 and the scale of the endoscopic image 40A are matching.

The notification message 164 is merely one example. For example, a notification may also be provided through sound or speech outputted from a speaker, or any other means of notification capable of making the user perceive that the scale of the reference image 150 and the scale of the endoscopic image 40A are matching.

On the other hand, under the second assumption, the second control unit 106B may simply calculate the difference between the value obtained by dividing the pixel count 156 by the transverse (or longitudinal) length of the characteristic portion 152 and the value obtained by dividing the pixel count 156 by the transverse (or longitudinal) length of the corresponding characteristic portion 154, and derive the assistance information 162 in a manner similar to the above. Also, in this case, the notification using the assistance information 162 and the notification that the scale of the reference image 150 and the scale of the endoscopic image 40A are matching may also be provided in a manner similar to the above.

The description herein gives an example of processing to make the scale of the endoscopic image 40A consistent with the scale of the reference image 150, but the technology of the present disclosure is not limited thereto. For example, processing to make optical features (such as color and luminance) of the endoscopic image 40A consistent with optical features of the reference image 150 may also be performed. In this case, for example, the second control unit 106B may simply calculate the difference between the optical features of the characteristic portion 152 and the optical features of the corresponding characteristic portion 154, and derive assistance information (for example, information indicating specific instructions regarding the type of light source and/or light intensity) to bring the calculated difference to zero. Also, in this case, the notification using the assistance information and the notification that the optical features of the reference image 150 and the optical features of the endoscopic image 40A are matching may also be provided in a manner similar to the above.

Also, in the example illustrated in FIG. 19, the assistance information 162 is derived through a comparison of the characteristic portion 152 and the corresponding characteristic portion 154, but the assistance information 162 may also be derived through a comparison of corresponding portions in the reference image 150 and the endoscopic image 40A other than the characteristic portion 152 and the corresponding characteristic portion 154.

Note that the scale of the reference image 150 and the optical features of the reference image 150 are an example of “imaging conditions of the first endoscopic examination” according to the technology of the present disclosure. Also, the scale of the endoscopic image 40A and the optical features of the endoscopic image 40A are an example of “imaging conditions of the second endoscopic examination” according to the technology of the present disclosure.

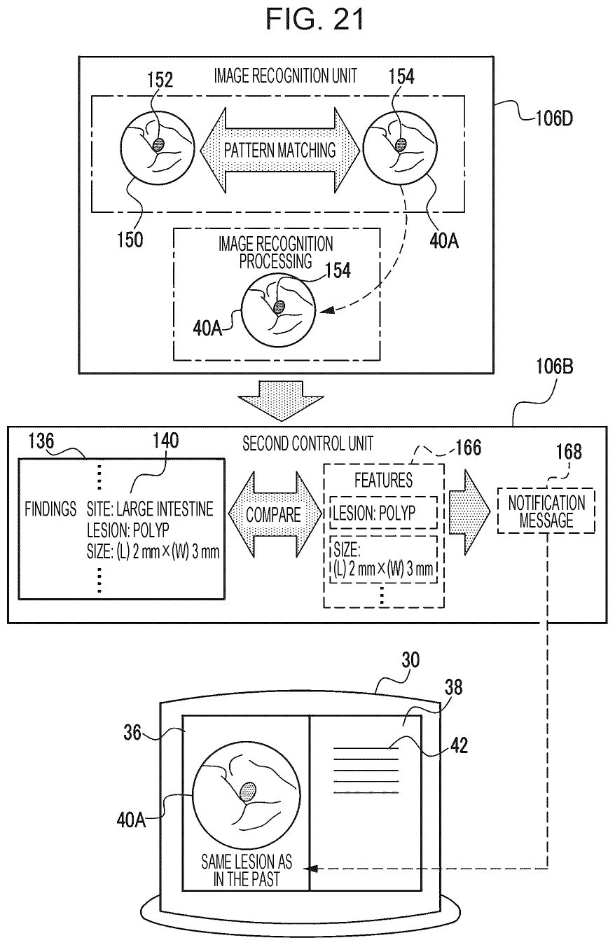

Upon completion of the processing to make the scale of the endoscopic image 40A consistent with the scale of the reference image 150 as described above, the processor 106 uses the reference image 150 and the endoscopic image 40A as a basis for determining whether or not the reference image 150 and the endoscopic image 40A show a common lesion. In this case, as illustrated by way of example in FIG. 21, the image recognition unit 106D identifies the corresponding characteristic portion 154 that matches the characteristic portion 152 by performing pattern matching between the reference image 150 and the endoscopic image 40A. The image recognition unit 106D then executes image recognition processing on the corresponding characteristic portion 154 to acquire features 166 of the corresponding characteristic portion 154. Examples of the features 166 include the type of lesion and size of lesion.

The second control unit 106B compares the features 166 with the endoscopic examination information 140 recorded in the report 136 to determine whether or not the endoscopic examination information 140 includes the same information as the features 166. In the case of determining that the endoscopic examination information 140 includes the same information as the features 166, the second control unit 106B generates a notification message 168. The notification message 168 is a message notifying that the reference image 150 and the endoscopic image 40A are determined to contain a common lesion. That is, the notification message 168 is a message notifying that there is a high likelihood that the same lesion as the lesion found in the first endoscopic examination has also been found in the second endoscopic examination. The second control unit 106B displays the notification message 168 on the screen 36 of the display apparatus 30.

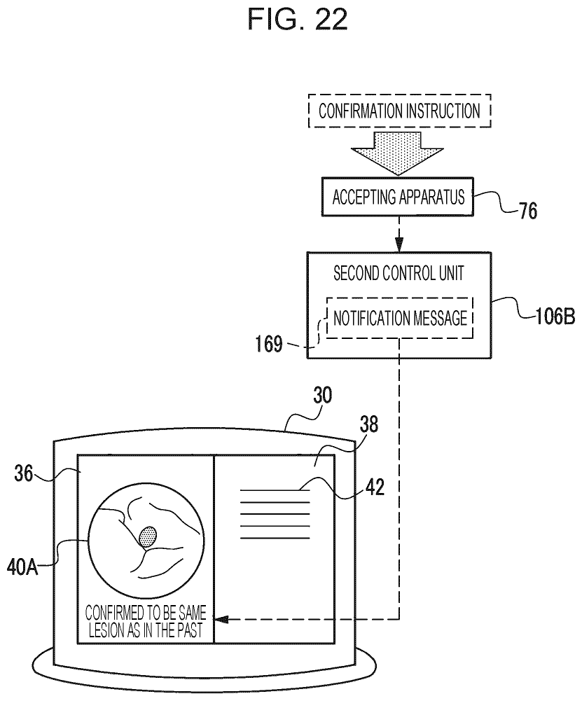

When the notification message 168 is displayed on the screen 36, the endoscopic image 40A and the notification message 168 are confirmed by the physician 14. Then, as illustrated by way of example in FIG. 22, the accepting apparatus 76 accepts a confirmation instruction from the physician 14. The confirmation instruction is an instruction to confirm the determination result indicating that the reference image 150 and the endoscopic image 40A contain a common lesion. When the confirmation instruction is accepted by the accepting apparatus 76, the second control unit 106B generates a notification message 169. The notification message 169 is a message providing a notification of confirmation of the determination result indicating that the reference image 150 and the endoscopic image 40A contain a common lesion. The second control unit 106B displays the notification message 169 on the screen 36 of the display apparatus 30. This provides a notification of confirmation of the determination result indicating that the reference image 150 and the endoscopic image 40A contain a common lesion. Note that the processing to provide a notification of confirmation of the determination result indicating that the reference image 150 and the endoscopic image 40A contain a common lesion is an example of “fourth notification processing” according to the technology of the present disclosure.

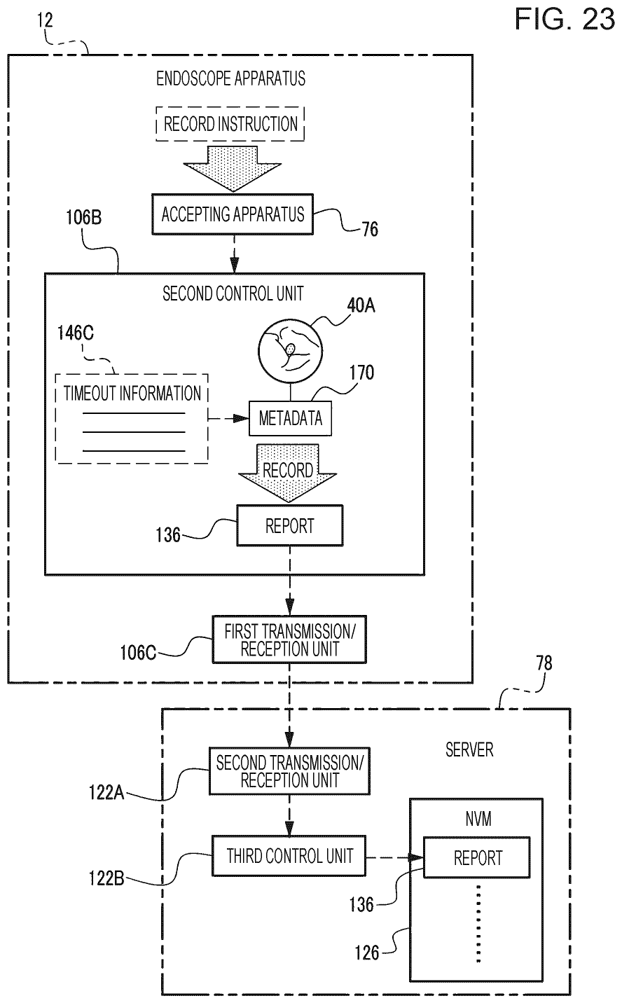

As illustrated by way of example in FIG. 23, the timeout information 146C obtained in a timeout of the second endoscopic examination is associated with the endoscopic image 40A. For example, processing to associate the timeout information 146C with the endoscopic image 40A is performed when the accepting apparatus 76 accepts a record instruction from the user. The record instruction is an instruction to record the timeout information 146C in the report 136. The second control unit 106B assigns metadata 170 to the endoscopic image 40A subjected to the image recognition processing illustrated in FIG. 21. When the accepting apparatus 76 accepts the record instruction, the second control unit 106B associates the timeout information 146C with the endoscopic image 40A by including the timeout information 146C in the metadata 170. The second control unit 106B records the endoscopic image 40A and the metadata 170 in the report 136. The report 136 in which the endoscopic image 40A and the metadata 170 are recorded is the report 136 received by the first transmission/reception unit 106C in the example illustrated in FIG. 12.

The report 136 in which the endoscopic image 40A and the metadata 170 are recorded is transmitted to the server 78 by the first transmission/reception unit 106C. In the server 78, the second transmission/reception unit 122A receives the report 136 transmitted from the first transmission/reception unit 106C. The third control unit 122B stores the report 136 received by the second transmission/reception unit 122A in the NVM 126.

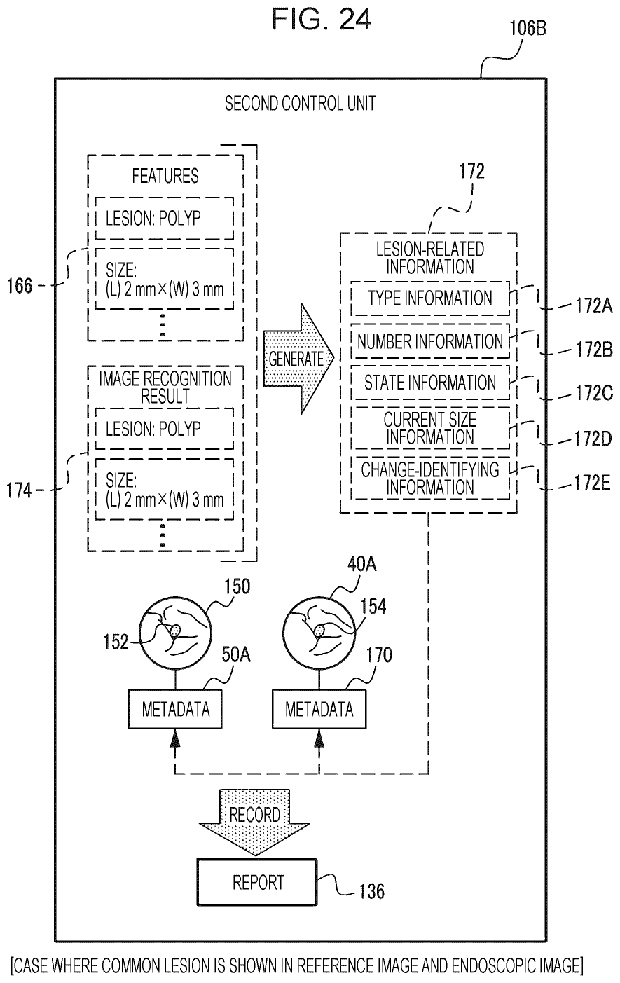

As illustrated by way of example in FIG. 24, when the reference image 150 and the endoscopic image 40A show a common lesion, the second control unit 106B generates lesion-related information 172 pertaining to the lesion (that is, the lesion common to the reference image 150 and the endoscopic image 40A). The lesion-related information 172 is generated on the basis of the features 166, which are obtained by performing the image recognition processing illustrated in FIG. 21 on the endoscopic image 40A, and an image recognition result 174. The image recognition result 174 is the result of the image recognition processing performed on the reference image 150 illustrated in FIG. 17. The image recognition result 174 includes the type of lesion, size of lesion, and the like.

The lesion-related information 172 includes type information 172A, number information 172B, and state information 172C. The lesion-related information 172 also includes current size information 172D and change-identifying information 172E as information pertaining to the size of lesion.

The type information 172A is information that can be used to identify the type of lesion common to the reference image 150 and the endoscopic image 40A. The number information 172B is information that can be used to identify the number of lesions common to the reference image 150 and the endoscopic image 40A. The state information 172C is information indicating the state (for example, the degree of inflammation, degree of bleeding, color of lesion, and/or shape of lesion) of the lesion common to the reference image 150 and the endoscopic image 40A. The state of lesion refers to the current state of a lesion, for example. The current state of lesion is identified from the features 166. The current size information 172D is information indicating the current size of the lesion common to the reference image 150 and the endoscopic image 40A. The current size is identified from the features 166. The change-identifying information 172E is information that can be used to identify change over time in the lesion common to the reference image 150 and the endoscopic image 40A.

The change-identifying information 172E is derived on the basis of the size of lesion shown in the reference image 150 and/or the size of lesion shown in the endoscopic image 40A. The size of lesion shown in the reference image 150 is the size of lesion included in the image recognition result 174, and the size of lesion shown in the endoscopic image 40A is the size of lesion included in the features 166. The number information 172B may be, for example, at least one piece of information from among the first to fourth examples given below.

The first example of the change-identifying information 172E is the ratio of the size of lesion shown in the endoscopic image 40A to the size of lesion shown in the reference image 150.

The second example of the change-identifying information 172E is the rate of change in the size of lesion. The rate of change in the size of lesion is the ratio of the elapsed time to a value obtained by subtracting the size of lesion shown in the reference image 150 from the size of lesion shown in the endoscopic image 40A. The elapsed time refers to the time elapsed from the date and time when the first endoscopic examination was performed to the date and time when the second endoscopic examination was performed, for example. The date and time when the first endoscopic examination was performed can be identified from the examination date and time (see FIG. 11) included in the endoscopic examination information 140 recorded in the report 136 received by the first transmission/reception unit 106C illustrated in FIG. 10, for example.

The third example of the change-identifying information 172E is information indicating the size of lesion after several days. The size of lesion after several days is identified by regression analysis (extrapolation, for example) using the size of lesion shown in the reference image 150 and the size of lesion shown in the endoscopic image 40A. The size of lesion after several days may also be a value obtained by multiplying the size identified by regression analysis by a predetermined coefficient for the type of lesion identified from the features 166 or the image recognition result 174.

The fourth example of the change-identifying information 172E is information indicating the size of lesion on a designated date and time. The size of lesion on a designated date and time may be derived using, for example, an arithmetic expression in which the size of lesion shown in the reference image 150 (and/or the size of lesion shown in the endoscopic image 40A), the type of lesion, and the time elapsed from the examination date and time when the first endoscopic examination (or the second endoscopic examination) was performed to the designated date and time are independent variables and the size of the lesion on the designated date and time is the dependent variable.

Note that the lesion-related information 174 is an example of “lesion-related information” according to the technology of the present disclosure. The current size information 172D and the change-identifying information 172E are an example of “size-related information” according to the technology of the present disclosure. The change-identifying information 172E is an example of “change-identifying information” according to the technology of the present disclosure. The type information 172A is an example of “information that can be used to identify the type of lesion” according to the technology of the present disclosure. The number information 172B is an example of “information that can be used to identify the number of lesions” according to the technology of the present disclosure. The state information 172C is an example of “information that can be used to identify the state of lesion” according to the technology of the present disclosure.

The second control unit 106B associates the lesion-related information 172 with the reference image 150 and the endoscopic image 40A by including the lesion-related information 172 in the metadata 50A and 170. Note that, as described in the example illustrated in FIG. 23, the metadata 170 also includes the timeout information 146C.

The second control unit 106B creates the report 136 in which the reference image 150, the endoscopic image 40A, and the lesion-related information 172 are recorded. That is, the second control unit 106B records the reference image 150 and the metadata 50A in the report 136. The report 136 in which the reference image 150 and the metadata 50A are recorded is stored in the NVM 126 of the server 78 in a manner similar to the example illustrated in FIG. 23. Also, the second control unit 106B records the endoscopic image 40A and the metadata 170 in the report 136. The report 136 in which the endoscopic image 40A and the metadata 170 are recorded is stored in the NVM 126 of the server 78 in a manner similar to the example illustrated in FIG. 23.

Note that the description herein gives an example in which the lesion-related information 172 is recorded in the report 136 by being included in the metadata 50A and 170, but the technology of the present disclosure is not limited thereto. For example, the lesion-related information 172 may also be recorded as part of the findings in the report 136.

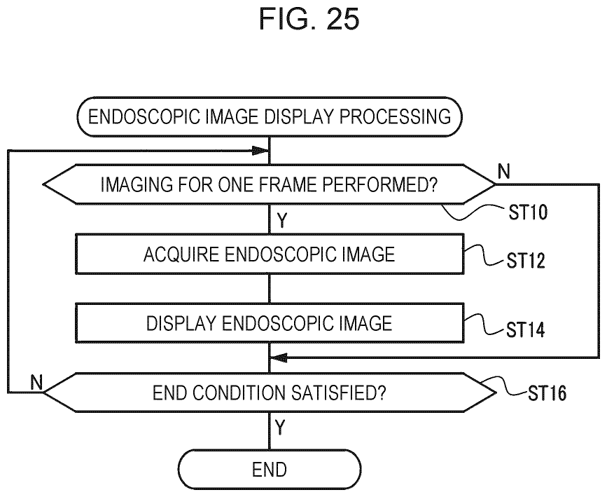

Next, the operation of the endoscope system 10 will be described with reference to FIGS. 25-27.

First, an example of the flow of the endoscopic image display processing performed by the processor 106 of the control apparatus 28 when the camera 66 is inserted into the large intestine 88 of the subject 20 in the second endoscopic examination will be described with reference to FIG. 25. Note that the following description assumes that endoscopic images 40A are sequentially acquired by the processor 106 due to the camera 66 performing imaging at a predetermined frame rate.

In the endoscopic image display processing illustrated in FIG. 25, first, in step ST10, the first control unit 106A determines whether or not the camera 66 has performed imaging for one frame. In step ST10, if the camera 66 has not performed imaging for one frame, the determination is negative, and the endoscopic image display processing proceeds to step ST16. In step ST10, if the camera 66 has performed imaging for one frame, the determination is positive, and the endoscopic image display processing proceeds to step ST12.

In step ST12, the first control unit 106A acquires the endoscopic image 40A obtained due to the camera 66 performing imaging for one frame (see FIG. 9). After the processing in step ST12 is executed, the endoscopic image display processing proceeds to step ST14.

In step ST14, the first control unit 106A displays the endoscopic image 40A acquired in step ST12 on the screen 36 (see FIG. 9). After the processing in step ST14 is executed, the endoscopic image display processing proceeds to step ST16.

In step ST16, the first control unit 106A determines whether or not a condition for ending the endoscopic image display processing (hereinafter referred to as the “endoscopic image display processing end condition”) is satisfied. The endoscopic image display processing end condition may be a condition stipulating that the accepting apparatus 76 has accepted an instruction to end the endoscopic image display processing. In step ST16, if the endoscopic image display processing end condition is not satisfied, the determination is negative, and the endoscopic image display processing proceeds to step ST10. In step ST16, if the endoscopic image display processing end condition is satisfied, the determination is positive, and the endoscopic image display processing ends.

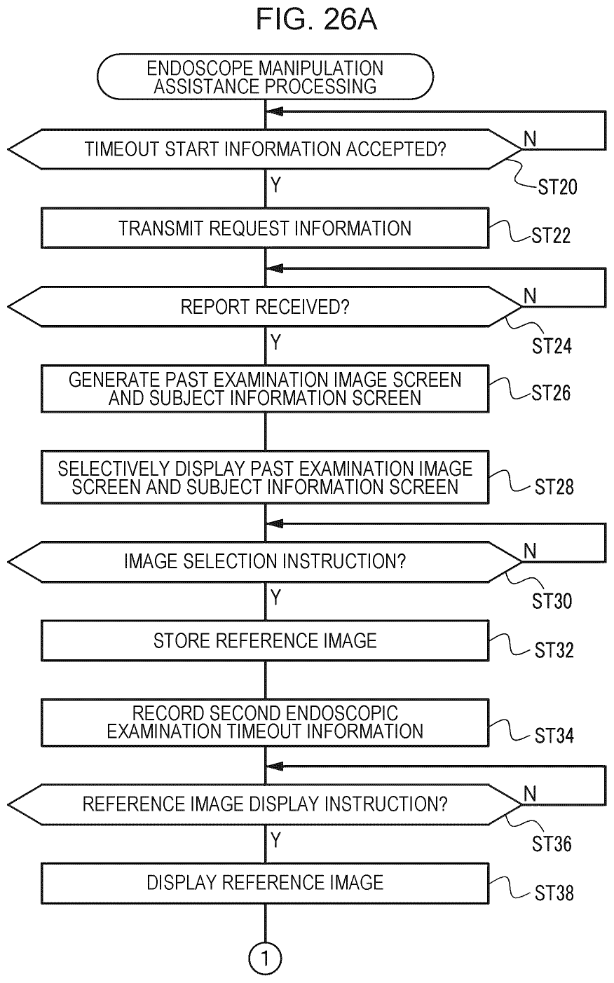

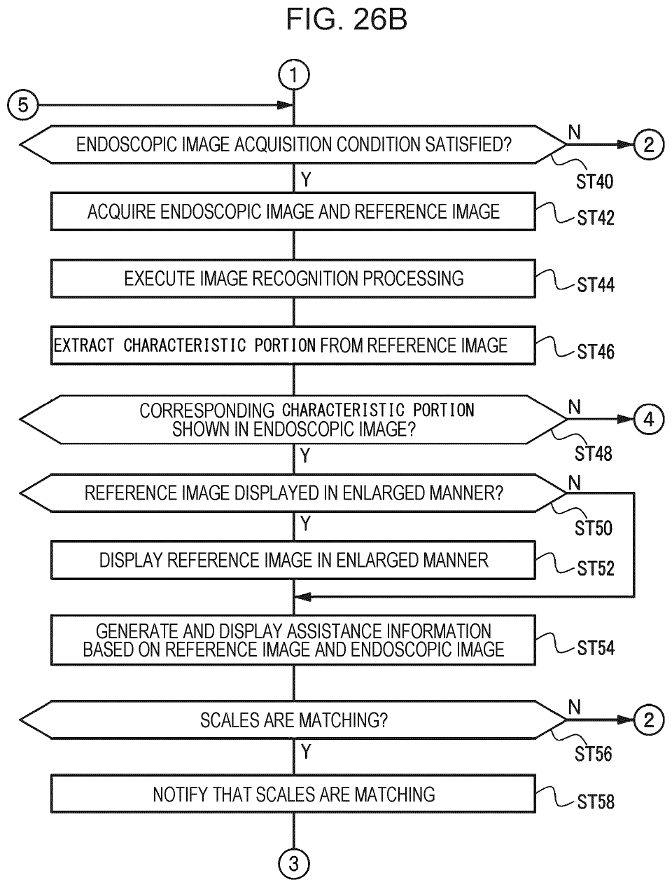

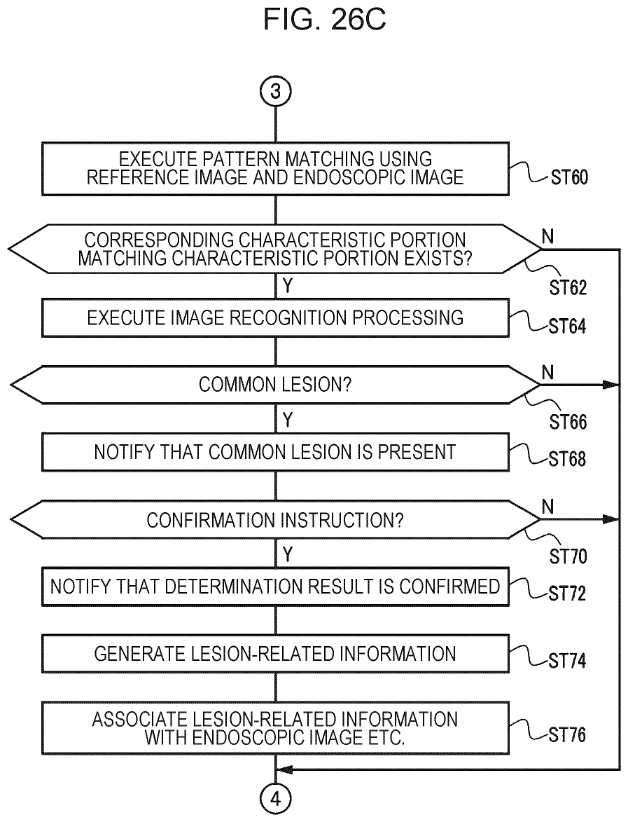

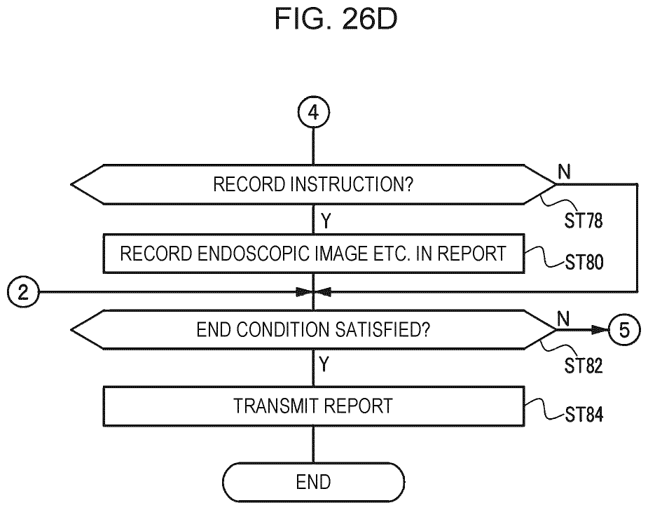

Next, an example of the flow of endoscope manipulation assistance processing performed by the processor 106 of the control apparatus 28 in the second endoscopic examination will be described with reference to FIGS. 26A-26D. Note that the flow of the endoscope manipulation assistance processing illustrated in FIGS. 26A-26D is an example of an “information processing method” according to the technology of the present disclosure. Also, the following description assumes that the endoscope manipulation assistance processing is performed in parallel with the endoscopic image display processing.

In the endoscope manipulation assistance processing illustrated in FIG. 26A, first, in step ST20, the first transmission/reception unit 106C determines whether or not the accepting apparatus 76 has accepted timeout start information (see FIG. 12). In step ST20, if the accepting apparatus 76 has not accepted timeout start information, the determination is negative and the determination in step ST20 is made again. In step ST20, if the accepting apparatus 76 has accepted timeout start information, the determination is positive, and the endoscope manipulation assistance processing proceeds to step ST22.

In step ST22, the first transmission/reception unit 106C transmits the request information 144 to the server 78 (see FIG. 12). After the processing in step ST22 is executed, the endoscope manipulation assistance processing proceeds to step ST24.

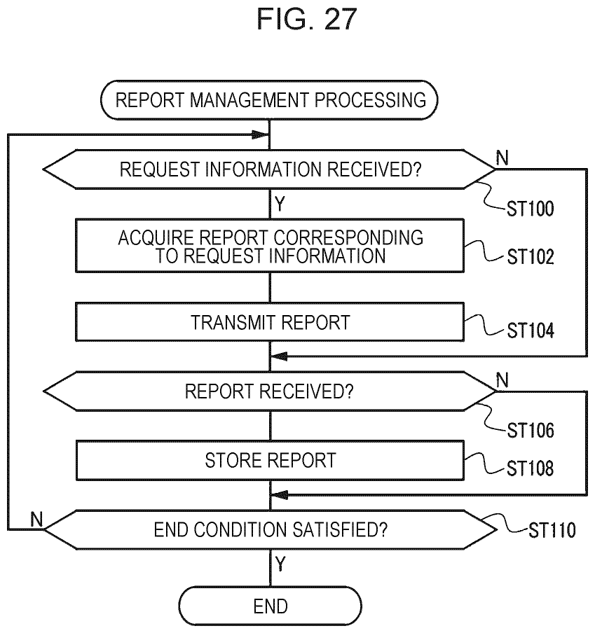

When the request information 144 is transmitted from the first transmission/reception unit 106C to the server 78 due to the execution of the processing in step ST22, the processing in step ST104 of the report management processing illustrated in FIG. 27 is executed in response. This causes the report 136 to be transmitted from the server 78 to the control apparatus 28 (see FIG. 12).

Accordingly, in step ST24, the second control unit 106B determines whether or not the first transmission/reception unit 106C has received the report 136 transmitted from the server 78 to the control apparatus 28. In step ST24, if the first transmission/reception unit 106C has not received the report 136, the determination is negative and the determination in step ST24 is made again. In step ST24, if the first transmission/reception unit 106C has received the report 136, the determination is positive, and the endoscope manipulation assistance processing proceeds to step ST26.

In step ST26, the second control unit 106B uses the report 136 received by the first transmission/reception unit 106C as a basis for generating the past examination image screen 46 and the subject information screen 146 (see FIG. 13). After the processing in step ST26 is executed, the endoscope manipulation assistance processing proceeds to step ST28.

In step ST28, the second control unit 106B causes the touch panel display 44 to selectively display the past examination image screen 46 and the subject information screen 146 generated in step ST26, according to a screen switching instruction accepted by the accepting apparatus 76 (see FIG. 14). After the processing in step ST28 is executed, the endoscope manipulation assistance processing proceeds to step ST30.

In step ST30, the second control unit 106B determines whether or not the touch panel display 44 has accepted an image selection instruction (see FIG. 15). In step ST30, if the touch panel display 44 has not accepted an image selection instruction, the determination is negative and the determination in step ST30 is made again. In step ST30, if the touch panel display 44 has accepted an image selection instruction, the determination is positive, and the endoscope manipulation assistance processing proceeds to step ST32.

In step ST32, the second control unit 106B acquires an examination result image 50 selected according to the image selection instruction as the reference image 150. The second control unit 106B also acquires the metadata 50A of the examination result image 50 selected according to the image selection instruction. The second control unit 106B then stores the reference image 150 and the metadata 50A in the reference image storage area 108A (see FIG. 15). After the processing in step ST32 is executed, the endoscope manipulation assistance processing proceeds to step ST34.

In step ST34, the second control unit 106B causes the timeout information 146C obtained in the timeout of the second endoscopic examination to be recorded in the report 136 and displayed on the subject information screen 146 of the touch panel display 44. After the processing in step ST34 is executed, the endoscope manipulation assistance processing proceeds to step ST36.

In step ST36, the second control unit 106B determines whether or not the accepting apparatus 76 has accepted a reference image display instruction (see FIG. 17). In step ST36, if the accepting apparatus 76 has not accepted a reference image display instruction, the determination is negative and the determination in step ST36 is made again. In step ST36, if the accepting apparatus 76 has accepted a reference image display instruction, the determination is positive, and the endoscope manipulation assistance processing proceeds to step ST38.

In step ST38, the second control unit 106B displays the reference image 150 in a manner distinguishable from the other examination result images 50 on the past examination image screen 46 of the touch panel display 44 (see the upper illustration in FIG. 18). This allows for comparison between the reference image 150 displayed on the past examination image screen 46 of the touch panel display 44 and the endoscopic image 40A, which is displayed on the screen 36 of the display apparatus 30 due to the execution of the processing in step ST14 illustrated in FIG. 25. After the processing in step ST38 is executed, the endoscope manipulation assistance processing proceeds to step ST40 illustrated in FIG. 26B.

In step ST40, the second control unit 106B determines whether or not a condition for acquiring the endoscopic image 40A (hereinafter referred to as the “endoscopic image acquisition condition”) is satisfied. A first example of the endoscopic image acquisition condition is a condition stipulating that the accepting apparatus 76 has accepted an instruction to acquire the endoscopic image 40A from the camera 66. A second example of the endoscopic image acquisition condition is a condition stipulating that a timing has been reached, the timing being designated in advance as the timing at which to acquire the endoscopic image 40A from the camera 66.

In step ST40, if the endoscopic image acquisition condition is not satisfied, the determination is negative, and the endoscope manipulation assistance processing proceeds to step ST82 illustrated in FIG. 26D. In step ST40, if the endoscopic image acquisition condition is satisfied, the determination is positive, and the endoscope manipulation assistance processing proceeds to step ST42.

In step ST42, the image recognition unit 106D acquires the endoscopic image 40A from the camera 66 (see FIG. 17). Also, the image recognition unit 106D acquires the reference image 150 from the reference image storage area 108A (see FIG. 17). After the processing in step ST42 is executed, the endoscope manipulation assistance processing proceeds to step ST44.

In step ST44, the image recognition unit 106D executes image recognition processing on the reference image 150 acquired from the reference image storage area 108A in step ST42 to identify the characteristic portion 152 in the reference image 150. After the processing in step ST44 is executed, the endoscope manipulation assistance processing proceeds to step ST46. In step ST46, the image recognition unit 106D extracts the characteristic portion 152 identified in step ST44 from the reference image 150. After the processing in step ST46 is executed, the endoscope manipulation assistance processing proceeds to step ST48.

In step ST48, the image recognition unit 106D compares the endoscopic image 40A acquired from the camera 66 in step ST42 with the characteristic portion 152 extracted from the reference image 150 in step ST46 to determine whether or not the corresponding characteristic portion 154 is shown in the endoscopic image 40A (see FIG. 17). In step ST48, if the corresponding characteristic portion 154 is not shown in the endoscopic image 40A, the determination is negative, and the endoscope manipulation assistance processing proceeds to step ST78 illustrated in FIG. 26D. In step ST48, if the corresponding characteristic portion 154 is shown in the endoscopic image 40A, the determination is positive, and the endoscope manipulation assistance processing proceeds to step ST50.

In step ST50, the second control unit 106B determines whether or not the reference image 150 is being displayed in an enlarged manner on the past examination image screen 46 of the touch panel display 44. In step ST50, if the reference image 150 is being displayed in an enlarged manner on the past examination image screen 46 of the touch panel display 44, the determination is negative, and the endoscope manipulation assistance processing proceeds to step ST54. In step ST50, if the reference image 150 is not being displayed in an enlarged manner on the past examination image screen 46 of the touch panel display 44, the determination is positive, and the endoscope manipulation assistance processing proceeds to step ST52.

In step ST52, the second control unit 106B displays the reference image 150 in an enlarged manner on the past examination image screen 46 of the touch panel display 44 (sec the lower illustration in FIG. 18). After the processing in step ST52 is executed, the endoscope manipulation assistance processing proceeds to step ST54.

In step ST54, the second control unit 106B generates assistance information 162 on the basis of the endoscopic image 40A and the reference image 150 acquired in step ST42 (see FIG. 19). The second control unit 106B then displays the generated assistance information 162 on the screen 36 of the display apparatus 30. After the processing in step ST54 is executed, the endoscope manipulation assistance processing proceeds to step ST56.

The physician 14 adjusts the position of the camera 66 while referring to the assistance information 162 displayed on the screen 36 due to the execution of the processing in step ST54.

In step ST56, the second control unit 106B determines whether or not the scale of the endoscopic image 40A matches the scale of the reference image 150. In step ST56, if the scale of the endoscopic image 40A does not match the scale of the reference image 150, the determination is negative, and the endoscope manipulation assistance processing proceeds to step ST82 illustrated in FIG. 26D. In step ST56, if the scale of the endoscopic image 40A matches the scale of the reference image 150, the determination is positive, and the endoscope manipulation assistance processing proceeds to step ST58.

In step ST58, the second control unit 106B performs processing to provide a notification that the scale of the reference image 150 and the scale of the endoscopic image 40A are matching by generating a notification message 164 and displaying the generated notification message 164 on the screen 36 (see FIG. 20). After the processing in step ST58 is executed, the endoscope manipulation assistance processing proceeds to step ST60 illustrated in FIG. 26C.

In step ST60 illustrated in FIG. 26C, the image recognition unit 106D executes pattern matching using the reference image 150 acquired in step ST42 and the scale-adjusted endoscopic image 40A (see FIG. 21). After the processing in step ST60 is executed, the endoscope manipulation assistance processing proceeds to step ST62.

In step ST62, the image recognition unit 106D uses the result of the pattern matching performed in step ST60 as a basis for determining whether or not a corresponding characteristic portion 154 matching the characteristic portion 152 in the reference image 150 is shown in the endoscopic image 40A. In step ST62, if a corresponding characteristic portion 154 matching the characteristic portion 152 in the reference image 150 is not shown in the endoscopic image 40A, the determination is negative, and the endoscope manipulation assistance processing proceeds to step ST78 illustrated in FIG. 26D. In step ST62, if a corresponding characteristic portion 154 matching the characteristic portion 152 in the reference image 150 is shown in the endoscopic image 40A, the determination is positive, and the endoscope manipulation assistance processing proceeds to step ST64.

In step ST64, the image recognition unit 106D executes image recognition processing on the endoscopic image 40A used in the pattern matching in step ST60 (see FIG. 21). After the processing in step ST64 is executed, the endoscope manipulation assistance processing proceeds to step ST66.

In step ST66, the second control unit 106B compares the features 166 obtained due to the execution of the image recognition processing in step ST64 with the endoscopic examination information 140 recorded in the report 136 received by the first transmission/reception unit 106C in step ST24 to determine whether or not the reference image 150 and the endoscopic image 40A contain a common lesion. In step ST66, if the reference image 150 and the endoscopic image 40A do not contain a common lesion, the determination is negative, and the endoscope manipulation assistance processing proceeds to step ST78 illustrated in FIG. 26D. In step ST66, if the reference image 150 and the endoscopic image 40A contain a common lesion, the determination is positive, and the endoscope manipulation assistance processing proceeds to step ST68.

In step ST68, the second control unit 106B displays the notification message 168 on the screen 36 of the display apparatus 30, thereby providing a notification that the reference image 150 and the endoscopic image 40A are determined to contain a common lesion. After the processing in step ST68 is executed, the endoscope manipulation assistance processing proceeds to step ST70.

When the notification message 168 is displayed on the screen 36, the endoscopic image 40A and the notification message 168 are confirmed by the physician 14. Then, in step ST70, the second control unit 106B determines whether or not the accepting apparatus 76 has accepted a confirmation instruction from the physician 14. In step ST70, if the accepting apparatus 76 has not accepted a confirmation instruction from the physician 14, the determination is negative, and the endoscope manipulation assistance processing proceeds to step ST78 illustrated in FIG. 26D. In step ST70, if the accepting apparatus 76 has accepted a confirmation instruction from the physician 14, the determination is positive, and the endoscope manipulation assistance processing proceeds to step ST72.

In step ST72, the second control unit 106B displays the notification message 169 on the screen 36 of the display apparatus 30, thereby providing a notification of confirmation of the determination result indicating that the reference image 150 and the endoscopic image 40A contain a common lesion (see FIG. 22). After the processing in step ST72 is executed, the endoscope manipulation assistance processing proceeds to step ST74.

In step ST74, the second control unit 106B generates the lesion-related information 172 on the basis of the features 166 obtained due to the execution of the processing in step ST64 and the image recognition result 174 obtained due to the execution of the processing in step ST44 (see FIG. 24). After the processing in step ST74 is executed, the endoscope manipulation assistance processing proceeds to step ST76.

In step ST76, the second control unit 106B associates the lesion-related information 172 generated in step ST74 with the reference image 150 and the endoscopic image 40A by including the lesion-related information 172 in the metadata 50A and 170. After the processing in step ST76 is executed, the endoscope manipulation assistance processing proceeds to step ST78 illustrated in FIG. 26D.

In step ST78, the second control unit 106B determines whether or not the accepting apparatus 76 has accepted a record instruction (see FIG. 23). In step ST78, if the accepting apparatus 76 has not accepted a record instruction, the determination is negative, and the endoscope manipulation assistance processing proceeds to step ST82. In step ST78, if the accepting apparatus 76 has accepted a record instruction, the determination is positive, and the endoscope manipulation assistance processing proceeds to step ST80.

In step ST80, the second control unit 106B associates the timeout information 146C obtained in a timeout with the endoscopic image 40A by including the timeout information 146C in the metadata 170 (see FIG. 23). The second control unit 106B records the endoscopic image 40A and the metadata 170 in the report 136 received by the first transmission/reception unit 106C in step ST24. Also, the second control unit 106B records the metadata 50A made to include the lesion-related information 172 in step ST76 and the reference image 150 in the report 136 received by the first transmission/reception unit 106C in step ST24 (see FIG. 24). After the processing in step ST80 is executed, the endoscope manipulation assistance processing proceeds to step ST82.

In step ST82, the second control unit 106B determines whether or not a condition for ending the endoscope manipulation assistance processing (hereinafter referred to as the “endoscope manipulation assistance processing end condition”) is satisfied. The endoscope manipulation assistance processing end condition may be a condition stipulating that the accepting apparatus 76 has accepted an instruction to end the endoscope manipulation assistance processing. In step ST82, if the endoscope manipulation assistance processing end condition is not satisfied, the determination is negative, and the endoscope manipulation assistance processing proceeds to step ST40 illustrated in FIG. 26B. In step ST82, if the endoscope manipulation assistance processing end condition is satisfied, the determination is positive, and the endoscope manipulation assistance processing proceeds to step ST84.

In step ST84, the first transmission/reception unit 106C transmits the report 136 received in step ST24 to the server 78 (see FIG. 23). After the processing in step ST84 is executed, the endoscope manipulation assistance processing ends.

Next, an example of the flow of the report management processing performed by the processor 122 of the server 78 will be described with reference to FIG. 27.

In the report management processing illustrated in FIG. 27, first, in step ST100, the third control unit 122B determines whether or not the second transmission/reception unit 122A has received the request information 144 transmitted from the endoscope apparatus 12 due to the execution of step ST22 illustrated in FIG. 26A. In step ST100, if the second transmission/reception unit 122A has not received the request information 144, the determination is negative, and the report management processing proceeds to step ST106. In step ST100, if the second transmission/reception unit 122A has received the request information 144, the determination is positive, and the report management processing proceeds to step ST102.

In step ST102, the third control unit 122B acquires, from the NVM 126, the report 136 corresponding to the request information 144 received by the second transmission/reception unit 122A in step ST100 (see FIG. 12). After the processing in step ST102 is executed, the report management processing proceeds to step ST104.

In step ST104, the second transmission/reception unit 122A transmits the report 136 acquired from the NVM 126 in step ST102 to the endoscope apparatus 12 (see FIG. 12). After the processing in step ST104 is executed, the report management processing proceeds to step ST106.

In step ST106, the third control unit 122B determines whether or not the second transmission/reception unit 122A has received the report 136 transmitted from the endoscope apparatus 12 due to the execution of step ST84 illustrated in FIG. 26D. In step ST106, if the second transmission/reception unit 122A has not received the report 136, the determination is negative, and the report management processing proceeds to step ST110. In step ST106, if the second transmission/reception unit 122A has received the report 136, the determination is positive, and the report management processing proceeds to step ST108.

In step ST108, the third control unit 122B stores, in the NVM 126, the report 136 received by the second transmission/reception unit 122A in step ST106 (see FIG. 23). After the processing in step ST108 is executed, the report management processing proceeds to step ST110.

In step ST110, the third control unit 122B determines whether or not a condition for ending the report management processing (hereinafter referred to as the “report management processing end condition”) is satisfied. The report management processing end condition may be a condition stipulating that the accepting apparatus 76 or 84 has accepted an instruction to end the report management processing. In step ST110, if the report management processing end condition is not satisfied, the determination is negative, and the report management processing proceeds to step ST100. In step ST110, if the report management processing end condition is satisfied, the determination is positive, and the report management processing ends.

As described above, in the endoscope system 10, multiple examination result images 50 are acquired by the control apparatus 28 from the NVM 126 of the server 78 (see FIGS. 11 and 12). Additionally, during the period of carrying out the second endoscopic examination, an examination result image 50 selected from among the multiple examination result images 50 according to an instruction from the physician 14 is displayed as the reference image 150 on the past examination image screen 46 of the touch panel display 44 (see FIG. 18). In the second endoscopic examination, the endoscopic image 40A is displayed on the screen 36 of the display apparatus 30, and thus the user can compare the reference image 150 displayed on the past examination image screen 46 with the endoscopic image 40A displayed on the screen 36 of the display apparatus 30. This enables the user to understand the change from the site deemed the main examination target (the characteristic portion 152, for example) in the first endoscopic examination to the site deemed the main examination target (the corresponding characteristic portion 154, for example) in the second endoscopic examination.

Also, in the endoscope system 10, the reference image 150 is displayed on the past examination image screen 46 in the timeout phase performed during the period of carrying out the second endoscopic examination. Consequently, this enables the user to understand, in the timeout phase performed during the period of carrying out the second endoscopic examination, the change from the site deemed the main examination target (the characteristic portion 152, for example) in the first endoscopic examination to the site deemed the main examination target (the corresponding characteristic portion 154, for example) in the second endoscopic examination.

Also, in the endoscope system 10, the timeout information 146B is displayed on the subject information screen 146 of the touch panel display 44 in the timeout phase of the second endoscopic examination (see FIG. 16). The timeout information 146B is information obtained in a timeout that was performed during the period of carrying out the first endoscopic examination. Consequently, this enables the user to understand, in the timeout phase performed during the period of carrying out the second endoscopic examination, the timeout information 146B obtained in a timeout that was performed during the period of carrying out the first endoscopic examination.

Also, in the endoscope system 10, the subject-identifying information 138, being recorded in the report 136, is acquired from the server 78 as information required in a timeout performed during the period of carrying out the second endoscopic examination. The subject-identifying information 146A, which is the same information as the subject-identifying information 138, is then displayed on the subject information screen 146 of the touch panel display 44. Consequently, the subject-identifying information 138 can be provided rapidly to the user as information required in a timeout performed during the period of carrying out the second endoscopic examination, as compared to the case where the subject-identifying information 146A is obtained on the tablet terminal 32 and then the obtained subject-identifying information 146A is displayed on the touch panel display 44 of the tablet terminal 32.

Also, in the endoscope system 10, when a timeout performing during the period of carrying out the second endoscopic examination is completed, the report 136 used in the timeout is transmitted to the server 78 and stored in the NVM 126 of the server 78. Consequently, information (the subject-identifying information 146A, for example) recorded in the report 136 used in the timeout performed during the period of carrying out the second endoscopic examination can be utilized in a timeout performed during the period of carrying out an endoscopic examination to be performed after the second endoscopic examination (the next endoscopic examination, for example).

Also, in the endoscope system 10, the metadata 50A is associated with the examination result image 50 as data that accompanies the examination result image 50. The metadata 50A includes the same information as the timeout information 142 obtained in a timeout performed during the period of carrying out the first endoscopic examination. Consequently, the user who uses the examination result image 50 (the user who observes the examination result image 50, for example) can be provided with a service (the displaying of information pertaining to the timeout information 142, for example) using the timeout information 142 obtained in a timeout performed during the period of carrying out the first endoscopic examination as information related to the examination result image 50.

Also, in the endoscope system 10, the metadata 170 is associated with the endoscopic image 40A as data that accompanies the endoscopic image 40A. The metadata 170 includes the timeout information 146C obtained in a timeout of the second endoscopic examination (see FIG. 23). Consequently, the user who uses the endoscopic image 40A (the user who observes the endoscopic image 40A, for example) can be provided with a service (the displaying of information pertaining to the timeout information 146C, for example) using the timeout information 146C obtained in a timeout of the second endoscopic examination as information related to the endoscopic image 40A.

Also, in the endoscope system 10, an examination result image 50 selected from among the multiple examination result images 50 according to an instruction from the physician 14 is displayed as the reference image 150 on the past examination image screen 46 of the touch panel display 44 (sec FIGS. 15 and 18). Consequently, this enables the user to observe the examination result image 50 intended by the physician 14 as the reference image 150.

Also, in the endoscope system 10, when the image recognition unit 106D has determined that the endoscopic image 40A shows the corresponding characteristic portion 154, the second control unit 106B provides a notification that the corresponding characteristic portion 154 is shown in the endoscopic image 40A by displaying the reference image 150 in an enlarged manner on the past examination image screen 46 (see the lower illustration in FIG. 18). This enables the user to perceive that the location corresponding to the characteristic portion 152 in the reference image 150 is shown in the endoscopic image 40A displayed on the screen 36 of the display apparatus 30.

Also, in the endoscope system 10, the second control unit 106B performs processing to assist with making the imaging conditions of the second endoscopic examination consistent with the imaging conditions of the first endoscopic examination, on the basis of the reference image 150 and the endoscopic image 40A (see FIG. 19). This allows for improved accuracy in comparing the reference image 150 with the endoscopic image 40A, as compared to the case of not making the imaging conditions of the first endoscopic examination consistent with the imaging conditions of the second endoscopic examination.

Also, in the endoscope system 10, the assistance information 162 is outputted to the display apparatus 30 as information required to make the imaging conditions of the second endoscopic examination consistent with the imaging conditions of the first endoscopic examination, and the assistance information 162 is displayed on the screen 36 of the display apparatus 30 (see FIG. 19). This allows for assistance with the work of adjusting the imaging conditions of the second endoscopic examination to raise the accuracy in comparing the reference image 150 with the endoscopic image 40A.

Also, in the endoscope system 10, the assistance information 162 is derived on the basis of the result of comparing the characteristic portion 152 shown in the reference image 150 with the corresponding characteristic portion 154 shown in the endoscopic image 40A (see FIG. 19), and the derived assistance information 162 is displayed on the screen 36 of the display apparatus 30. Consequently, the imaging conditions used in the imaging of the characteristic portion 152 can be made consistent with the imaging conditions used in the imaging of the corresponding characteristic portion 154 in an accurate manner, as compared to the case where assistance information 162 derived without using the characteristic portion 152 and the corresponding characteristic portion 154 is displayed on the screen 36 of the display apparatus 30.

Also, in the endoscope system 10, a notification is provided when the imaging conditions of the second endoscopic examination and the imaging conditions of the first endoscopic examination are matching. For example, the notification message 164 is displayed on the screen 36 of the display apparatus 30 as a message notifying that the scale of the reference image 150 and the scale of the endoscopic image 40A are matching (see FIG. 20). Consequently, this enables the user to perceive that the imaging conditions of the second endoscopic examination and the imaging conditions of the first endoscopic examination are matching (for example, the scale of the reference image 150 and the scale of the endoscopic image 40A are matching).

Also, in the endoscope system 10, it is determined whether or not a common lesion is shown in the reference image 150 and the endoscopic image 40A, on the basis of the reference image 150 and the endoscopic image 40A (see FIG. 21). The notification message 168 is then displayed on the screen 36 of the display apparatus 30 as information indicating the determination result (see FIG. 21). Consequently, the user or the like (for example, the user and/or various apparatuses such as the control apparatus 28) can perform processing depending on whether or not a common lesion is shown in the reference image 150 and the endoscopic image 40A.

Also, in the endoscope system 10, if it is determined that a common lesion is shown in the reference image 150 and the endoscopic image 40A and the accepting apparatus 76 has accepted a confirmation instruction from the physician 14, the notification message 169 is displayed on the screen 36 of the display apparatus 30 to provide a notification of confirmation of the determination result indicating that a common lesion is shown in the reference image 150 and the endoscopic image 40A (see FIG. 22). This enables the user to perceive the confirmation of the determination result indicating that a common lesion is shown in the reference image 150 and the endoscopic image 40A.

Also, in the endoscope system 10, if a common lesion is shown in the reference image 150 and the endoscopic image 40A, the lesion-related information 172 is generated as information pertaining to the common lesion (see FIG. 24). With this arrangement, when a common lesion is shown in the reference image 150 and the endoscopic image 40A, the user or the like can perform processing based on the lesion-related information 172 generated as information pertaining to the common lesion.

Also, in the endoscope system 10, the lesion-related information 172 includes the current size information 172D and the change-identifying information 172E as information pertaining to the size of the lesion common to the reference image 150 and the endoscopic image 40A (see FIG. 24). With this arrangement, when a common lesion is shown in the reference image 150 and the endoscopic image 40A, the user or the like can perform processing based on the current size information 172D and the change-identifying information 172E generated as information pertaining to the size of the lesion common to the reference image 150 and the endoscopic image 40A.

Also, in the endoscope system 10, the lesion-related information 172 includes the change-identifying information 172E as information that can be used to identify change over time in the size of the lesion common to the reference image 150 and the endoscopic image 40A (see FIG. 24). This enables the user or the like to perform processing based on the change-identifying information 172E generated as information that can be used to identify change over time in the size of the lesion common to the reference image 150 and the endoscopic image 40A.

Also, in the endoscope system 10, the change-identifying information 172E is derived on the basis of the size of lesion shown in the reference image 150 and/or the size of lesion shown in the endoscopic image 40A. This allows for accurate derivation of the change-identifying information 172E as compared to the case of deriving the change-identifying information 172E solely from information unrelated to the size of lesion.

Also, in the endoscope system 10, the change-identifying information 172E is derived on the basis of the size of lesion shown in the reference image 150 and/or the size of lesion shown in the endoscopic image 40A, and the type of lesion. This allows for accurate derivation of the change-identifying information 172E as compared to the case of deriving the change-identifying information 172E without regard for the type of lesion.

Also, in the endoscope system 10, the lesion-related information 172 includes the type information 172A, the number information 172B, and the state information 172C (see FIG. 24). With this arrangement, when a common lesion is shown in the reference image 150 and the endoscopic image 40A, the user or the like can perform processing based on the type of lesion, number of lesions, and state of lesion.

Also, in the endoscope system 10, the lesion-related information 172 is associated with the reference image 150 and the endoscopic image 40A showing a common lesion (see FIG. 24). With this arrangement, when performing processing on the reference image 150, the user or the like can perform processing based on the lesion-related information 172 associated with the reference image 150. Also, when performing processing on the endoscopic image 40A, the user or the like can perform processing based on the lesion-related information 172 associated with the endoscopic image 40A.

Also, in the endoscope system 10, the report 136 in which the lesion-related information 172, the reference image 150, and the endoscopic image 40A are recorded is created (sec FIGS. 23 and 24). This allows the user or the like to identify the lesion-related information 172 and the reference image 150 that are in a correspondence relationship with each other through the report 136. This also allows the user or the like to identify the lesion- related information 172 and the endoscopic image 40A that are in a correspondence relationship with each other through the report 136.

Also, in the endoscope system 10, the reference image 150 is displayed on the touch panel display 44 of the tablet terminal 32 (see FIG. 18) and the endoscopic image 40A is displayed on the display apparatus 30 (see FIG. 9). The touch panel display 44 and the touch panel display 44 are arranged adjacently (sec FIG. 1). That is, the reference image 150 and the endoscopic image 40A are displayed in a manner allowing for comparison. Consequently, this enables the user to visually compare the reference image 150 and the endoscopic image 40A.

### First Modification

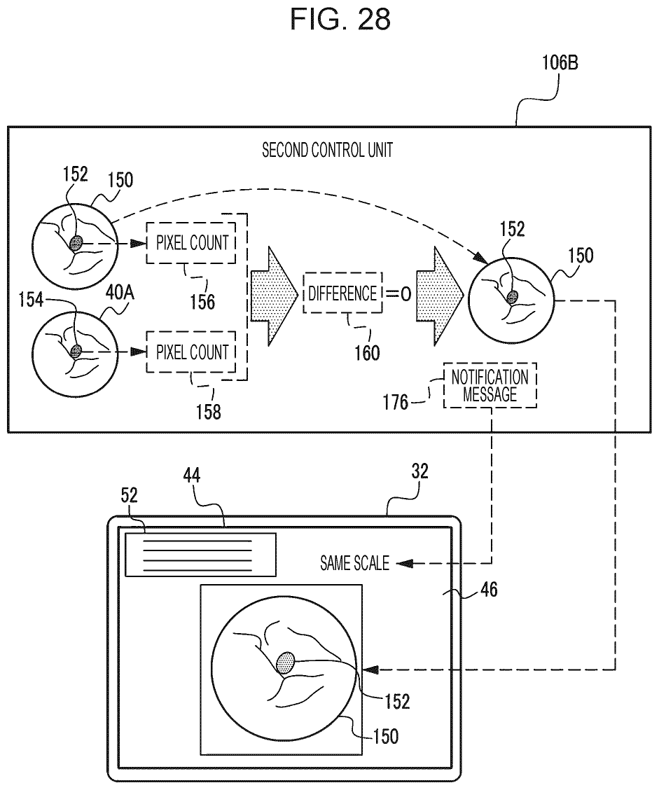

The embodiment above is described using an example in which the scale of the endoscopic image 40A is made consistent with the scale of the reference image 150 by adjusting the position of the camera 66 such that the difference 160 is zero, but the technology of the present disclosure is not limited thereto. For example, as illustrated in FIG. 28, the second control unit 106B may also change the display appearance of the reference image 150 according to the positional relationship between the characteristic portion 152 shown in the reference image 150 and the corresponding characteristic portion 154 shown in the endoscopic image 40A.

The example illustrated in FIG. 28 illustrates a situation in which the scale of the reference image 150 is changed such that the difference 160 is zero under the first assumption described in the embodiment above. Although the case where the difference 160 is zero under the first assumption is illustrated by way of example, this is merely one example, and the scale of the reference image 150 may also be changed such that the difference 160 is a value designated in advance as a value other than zero. Also, even when under the second assumption described in the embodiment above, a difference calculated in a manner similar to the embodiment above may be used to change the display appearance of the reference image 150 according to the positional relationship between the characteristic portion 152 shown in the reference image 150 and the corresponding characteristic portion 154 shown in the endoscopic image 40A.

Also, as illustrated by way of example in FIG. 28, the second control unit 106B generates a notification message 176 when the positional relationship between the characteristic portion 152 shown in the reference image 150 and the corresponding characteristic portion 154 shown in the endoscopic image 40A is a predetermined positional relationship (in this case, when the difference 160 is zero, as an example). The second control unit 106B then displays the generated notification message 176 on the past examination image screen 46. The notification message 176 is a message notifying that the positional relationship between the characteristic portion 152 shown in the reference image 150 and the corresponding characteristic portion 154 shown in the endoscopic image 40A is the predetermined positional relationship. Displaying the notification message 176 on the past examination image screen 46 provides a notification that the positional relationship between the characteristic portion 152 shown in the reference image 150 and the corresponding characteristic portion 154 shown in the endoscopic image 40A is the predetermined positional relationship. Note that the processing to provide a notification that the positional relationship between the characteristic portion 152 shown in the reference image 150 and the corresponding characteristic portion 154 shown in the endoscopic image 40A is the predetermined positional relationship is an example of “second notification processing” according to the technology of the present disclosure.

In this way, in the first modification, the display appearance of the reference image 150 is changed according to the positional relationship between the characteristic portion 152 shown in the reference image 150 and the corresponding characteristic portion 154 shown in the endoscopic image 40A. Consequently, alignment between the characteristic portion 152 shown in the reference image 150 and the corresponding characteristic portion 154 shown in the endoscopic image 40A can be achieved without requiring the physician 14 to operate the camera 66.

Also, in the first modification, the notification message 176 is displayed on the past examination image screen 46 to provide a notification that the positional relationship between the characteristic portion 152 shown in the reference image 150 and the corresponding characteristic portion 154 shown in the endoscopic image 40A is the predetermined positional relationship. Consequently, this enables the user to perceive that the positional relationship between the characteristic portion 152 shown in the reference image 150 and the corresponding characteristic portion 154 shown in the endoscopic image 40A is the predetermined positional relationship.

Note that although the first modification gives an example in which the scale of the reference image 150 is changed, it is also possible to make optical features of the reference image 150 consistent with optical features of the endoscopic image 40A.

### Second Modification

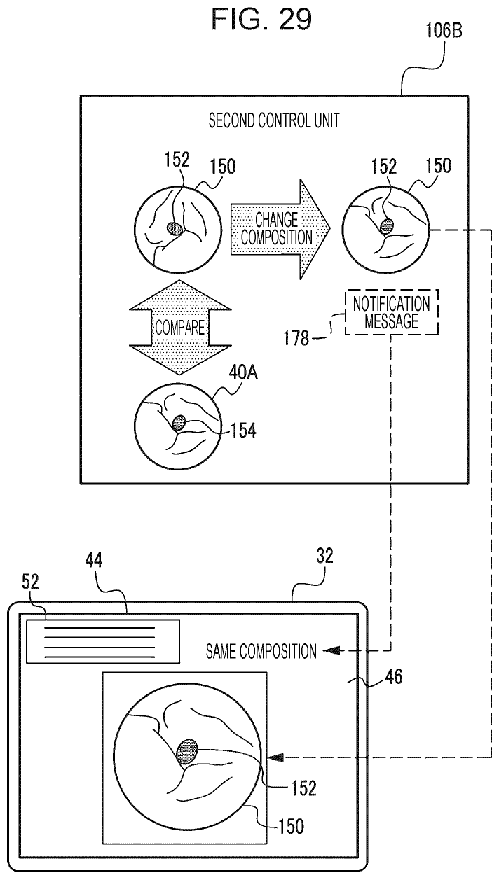

The first modification above is described using an example in which the scale of the reference image 150 is changed, but the technology of the present disclosure is not limited thereto. For example, as illustrated in FIG. 29, the second control unit 106B may also change the composition of the reference image 150 (such as the size and position of geometric elements included in the image) according to the endoscopic image 40A. In this case, for example, the second control unit 106B compares the endoscopic image 40A with the reference image 150 and uses the result of the comparison as a basis for changing the composition of the reference image 150 such that the composition of the reference image 150 matches the endoscopic image 40A. The reference image 150 with the changed composition is displayed on the past examination image screen 46 by the second control unit 106B.

Also, when the composition of the reference image 150 matches the endoscopic image 40A, the second control unit 106B generates a notification message 178 and displays the generated notification message 178 on the past examination image screen 46. The notification message 178 is a message notifying that the composition of the reference image 150 matches the endoscopic image 40A. Consequently, displaying the notification message 178 on the past examination image screen 46 provides a notification that the composition of the reference image 150 matches the endoscopic image 40A.

In this way, in the second modification, the composition of the reference image 150 is changed according to the endoscopic image 40A. Consequently, the user or the like can easily identify differences between the reference image 150 and the endoscopic image 40A, as compared to the case where the composition of the reference image 150 is fixed.

### Third Modification

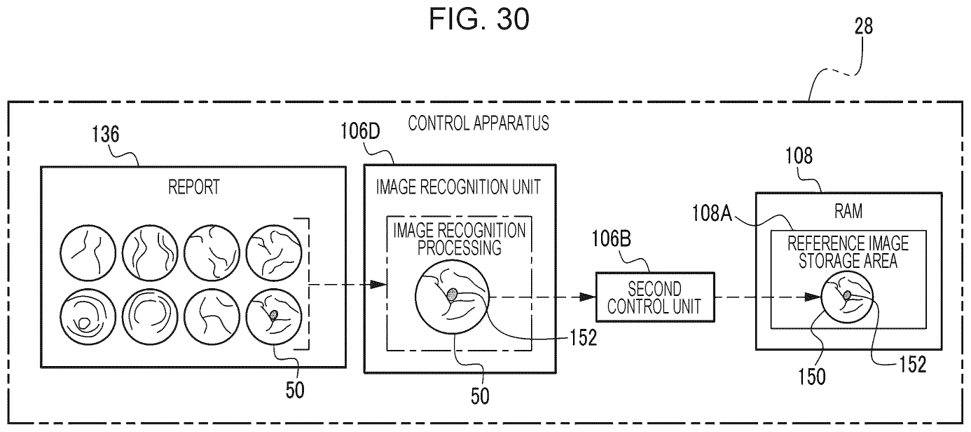

The embodiment above is described using an example in which the examination result image 50 selected according to the image selection instruction is handled as the reference image 150, which is an image satisfying a specific condition, but the technology of the present disclosure is not limited thereto. For example, as illustrated in FIG. 30, an examination result image 50 selected due to the image recognition unit 106D performing image recognition processing on multiple examination result images 50 recorded in the report 136 may also be handled in a manner similar to the embodiment above as the reference image 150, which is an image satisfying a specific condition. The example illustrated in FIG. 30 illustrates a situation in which image recognition processing is performed to select an examination result image 50 showing the characteristic portion 152 from among multiple examination result images 50, and the selected examination result image 50 is stored in the reference image storage area 108A as the reference image 150, which is an image satisfying a specific condition.

In this way, in the third modification, an examination result image 50 selected by performing image recognition processing on multiple examination result images 50 is handled in a manner similar to the embodiment above as the reference image 150, which is an image satisfying a specific condition. Consequently, an examination result image 50 showing the characteristic portion 152 is selected as the reference image 150, without requiring the physician 14 to select the examination result image 50 showing the characteristic portion 152 from among multiple examination result images 50.

Note that, in the third modification, an examination result image 50 selected by performing image recognition processing on multiple examination result images 50 is handled in a manner similar to the embodiment above as the reference image 150, which is an image satisfying a specific condition, but the technology of the present disclosure is not limited thereto. For example, an examination result image 50 selected by performing image recognition processing on multiple examination result images 50 may be displayed in a manner distinguishable from the other examination result images 50 on the past examination image screen 46, and when the examination result image 50 displayed in a distinguishable manner is selected according to the image selection instruction, the examination result image 50 selected according to the image selection instruction may be handled in a manner similar to the embodiment above as the reference image 150, which is an image satisfying a specific condition. In this case, the examination result image 50 showing the characteristic portion 152 intended by the physician 14 is selected as the reference image 150, without requiring the physician 14 to select the examination result image 50 showing the characteristic portion 152 from among multiple examination result images 50.

### Fourth modification

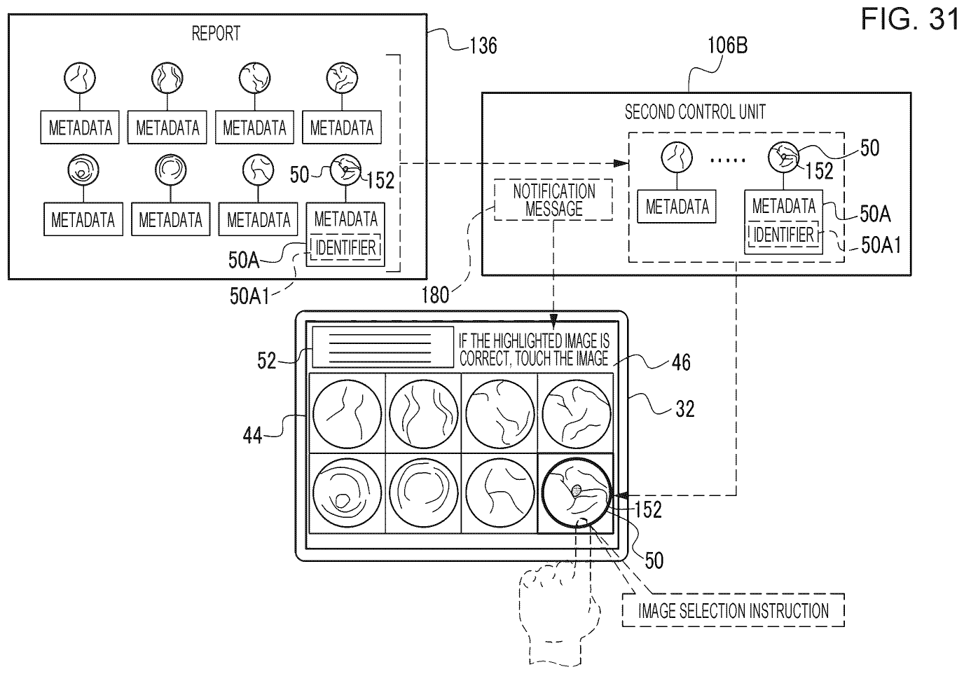

The third modification above is described using an example in which an examination result image 50 selected by performing image recognition processing on multiple examination result images 50 is selected as the reference image 150, which is an image satisfying a specific condition, but the technology of the present disclosure is not limited thereto. For example, as illustrated in FIG. 31, an examination result image 50 which is selected by performing information processing on the metadata 50A attached to each of multiple examination result images 50 recorded in the report 136 and which is selected according to the image selection instruction may also be selected as the reference image 150, which is an image satisfying a specific condition.

As illustrated by way of example in FIG. 31, the metadata 50A of an examination result image 50 showing the characteristic portion 152 contains an identifier 50A1. The identifier 50A1 is information that can be used to uniquely identify the examination result image 50 showing the characteristic portion 152. The second control unit 106B specifies the metadata 50A containing the identifier 50Al from among the multiple pieces of the metadata 50A corresponding to the multiple examination result images 50 recorded in the report 136. The second control unit 106B then displays the examination result image 50 to which the metadata 50A containing the identifier 50Al is attached on the past examination image screen 46, with a display appearance that is distinguishable from the other examination result images 50. The display appearance that is distinguishable from the other examination result images 50 may be, for example, a highlighted outline of the examination result image 50 to which the metadata 50A containing the identifier 50Al is attached. Also, the second control unit 106B displays a guidance message 180 on the past examination image screen 46. The guidance message 180 is a message asking the physician 14 whether or not the examination result image 50 displayed on the past examination image screen 46 with a display appearance distinguishable from the other examination result images 50 may be handled as the reference image 150.

If the examination result image 50 displayed on the past examination image screen 46 with a display appearance that is distinguishable from the other examination result images 50 is selected according to the image selection instruction, the examination result image 50 selected according to the image selection instruction is selected as the reference image 150, which is an image satisfying a specific condition.

In this way, in the fourth modification, an examination result image 50 which is selected by performing information processing on the metadata 50A attached to each of multiple examination result images 50 recorded in the report 136 and which is selected according to the image selection instruction is selected as the reference image 150, which is an image satisfying a specific condition. Consequently, the examination result image 50 showing the characteristic portion 152 intended by the physician 14 is selected as the reference image 150, without requiring the physician 14 to select the examination result image 50 showing the characteristic portion 152 from among multiple examination result images 50.

Note that in the example illustrated in FIG. 31, the examination result image 50 selected according to the image selection instruction is selected as the reference image 150, which is an image satisfying a specific condition, but this is merely one example. For example, the metadata 50A containing the identifier 50Al may be identified from among the multiple pieces of metadata 50A corresponding to the multiple examination result images 50 recorded in the report 136, and the examination result image 50 to which the identified metadata 50A is attached may be selected as the reference image 150, which is an image satisfying a specific condition. In this case, an examination result image 50 showing the characteristic portion 152 is selected as the reference image 150, without requiring the physician 14 to select the examination result image 50 showing the characteristic portion 152 from among multiple examination result images 50.

The metadata 50A may also contain the endoscopic examination information 140. In this case, the second control unit 106B performs information processing on the endoscopic examination information 140 in the metadata 50A to determine whether or not the examination result image 50 shows the characteristic portion 152. That is, if the endoscopic examination information 140 in the metadata 50A contains information related to a lesion corresponding to the characteristic portion 152, the second control unit 106B identifies that the examination result image 50 corresponding to the metadata 50A containing the information pertaining to a lesion corresponding to the characteristic portion 152 is an image showing the characteristic portion 152. The examination result image 50 corresponding to the metadata 50A containing the information pertaining to a lesion corresponding to the characteristic portion 152 is then selected as the reference image 150. In this case, an examination result image 50 showing the characteristic portion 152 is selected as the reference image 150, without requiring the physician 14 to select the examination result image 50 showing the characteristic portion 152 from among multiple examination result images 50.

Also, the second control unit 106B may identify the examination result image 50 corresponding to the metadata 50A containing the information pertaining to a lesion corresponding to the characteristic portion 152, and if the identified examination result image 50 is selected according to the image selection instruction, the examination result image 50 corresponding to the metadata 50A containing the information pertaining to a lesion corresponding to the characteristic portion 152 may be selected as the reference image 150. In this case, the examination result image 50 showing the characteristic portion 152 intended by the physician 14 is selected as the reference image 150, without requiring the physician 14 to select the examination result image 50 showing the characteristic portion 152 from among multiple examination result images 50.

Also, the metadata 50A may contain the same information as the timeout information 142 or part of the timeout information 142. In this case, the second control unit 106B performs information processing on the same information as the timeout information 142 or part of the timeout information 142 to determine whether or not the examination result image 50 shows the characteristic portion 152. That is, if the same information as the timeout information 142 or part of the timeout information 142 contains information related to a lesion corresponding to the characteristic portion 152, the second control unit 106B identifies that the examination result image 50 corresponding to the metadata 50A containing the information pertaining to a lesion corresponding to the characteristic portion 152 is an image showing the characteristic portion 152. Effects similar to the abovementioned are likewise obtained in this case.

### Other Modifications

The embodiment above is described using an example in which multiple examination result images 50 obtained in the first endoscopic examination are displayed on the past examination image screen 46, but the technology of the present disclosure is not limited thereto. For example, multiple types of past examination image screens 46 may also be displayed selectively according to a selection instruction by the user.

For example, in this case, the last endoscopic examination performed on the subject 20 and the second-to-last endoscopic examination (or an endoscopic examination earlier than second-to-last) performed on the subject 20 are selected by the user. Then, in response to a selection instruction by the user, the display is switched between a past examination image screen 46 containing multiple examination result images 50 obtained in the last endoscopic examination performed on the subject 20 and a past examination image screen 46 containing multiple examination result images 50 obtained in the second-to-last endoscopic examination (or an endoscopic examination earlier than second-to-last) performed on the subject 20. The display of multiple types of past examination image screens 46 may also be switched in response to an operation performed using voice recognition.

Multiple examination result images 50 obtained in the last endoscopic examination performed on the subject 20 and multiple examination result images 50 obtained in the second-to-last endoscopic examination (or an endoscopic examination earlier than second-to-last) performed on the subject 20 may also be displayed adjacently (in a manner allowing for comparison, for example).

The embodiment above is described using an example in which multiple examination result images 50 and the subject-identifying information 52 are displayed adjacently on the past examination image screen 46 (see FIG. 1 and FIGS. 13-15), but the technology of the present disclosure is not limited thereto. For example, the multiple examination result images 50 and the subject-identifying information 52 may also be displayed selectively according to a selection instruction by the user. Likewise in this case, the display may also be switched according to an operation performed using voice recognition.

The embodiment above is described using an example in which the control apparatus 28 acquires the subject-identifying information 138 and the endoscopic examination information 140 (see FIG. 11) from the server 78, but the technology of the present disclosure is not limited thereto. For example, if an instruction from the user is accepted by the tablet terminal 32 or the like on the endoscope apparatus 12 side before a timeout, the control apparatus 28 may acquire various information (such as the subject-identifying information 138 and the endoscopic examination information 140) from the server 78 and/or the tablet terminal 32 or the like, in response to the instruction from the user.

The embodiment above is described using an example in which the past examination image screen 46 is displayed on the tablet terminal 32, but the technology of the present disclosure is not limited thereto. For example, a monitor display button may be displayed on the touch panel display 44 of the tablet terminal 32, and when the monitor display button is turned on, a sub-screen corresponding to the screen displayed on the touch panel display 44 may also be displayed on the display apparatus 30 or the display appearance may be changed, in conjunction with changes to the content displayed on the touch panel display 44.

For example, a screen corresponding to the past examination image screen 46 (a screen showing a reduced version of the past examination image screen 46, for example) or part of the information included on the past examination image screen 46 (at least some of the multiple examination result images 50 and/or at least part of the subject-identifying information 52, for example) may also be displayed on the display apparatus 30, on the condition that the monitor display button displayed on the touch panel display 44 of the tablet terminal 32 is turned on. The location where the display apparatus 30 is to be displayed may be, for example, an edge of the screen (the right edge as seen from the front, for example) of the display apparatus 30.

Also, at least one of the multiple examination result images 50 (the reference image 150, for example) may also be displayed on the touch panel display 44 of the tablet terminal 32, on the condition that the monitor display button is turned on. Additionally, in conjunction with the above, the same examination result image 50 as the examination result image 50 displayed in an enlarged manner on the tablet terminal 32 side may also be displayed in an enlarged manner on the display apparatus 30. For example, when multiple examination result images 50 are displayed on the sub-screen of the display apparatus 30, the sub-screen is displayed in an enlarged manner. This causes the multiple examination result images 50 to be displayed in an enlarged manner on the display apparatus 30 side. Also, instead of changing the size of the sub-screen, only a specific examination result image 50 (the reference image 150, for example) within the sub-screen may be displayed in an enlarged manner. The sub-screen may also contain the subject-identifying information 52 or the like, and the display appearance of the subject-identifying information 52 or the like may also be changed in accordance with a change in the display appearance of the specific examination result image 50. In this case, for example, when the specific examination result image 50 is displayed in an enlarged manner, information (subject-identifying information 52 or the like) other than the specific examination result image 50 may be displayed in a reduced manner, may be displayed in an enlarged manner, or may not be displayed. Note that when the monitor display button is turned off, the original screens (the screens illustrated in FIG. 1, for example) are displayed on the display apparatus 30 and the touch panel display 44.

The embodiment above is described using an example in which the control apparatus processing is performed by the control apparatus 28, but the technology of the present disclosure is not limited thereto. For example, the device that performs the control apparatus processing may also be provided externally to the control apparatus 28. One example of a device provided externally to the control apparatus 28 is the server 78. The server 78 may also be realized by cloud computing. Cloud computing is merely one example, and the server 78 may also be realized by a mainframe, or by network computing such as fog computing, edge computing, or grid computing, for example.

Although the server 78 is given as an example of a device provided externally to the control apparatus 28, this is merely one example, and at least one personal computer or the like may be provided in place of the server 78. The control apparatus processing may also be performed in a distributed manner by multiple devices, including the control apparatus 28 and a device provided externally to the control apparatus 28. The device provided externally to the control apparatus 28 may also be the endoscopic processing apparatus 22.

The embodiment above is described using an example in which the endoscopic image 40A is displayed on the display apparatus 30 while the past examination image screen 46 and the subject information screen 146 are displayed on the touch panel display 44 of the tablet terminal 32, but the technology of the present disclosure is not limited thereto. For example, the endoscopic image 40A, the past examination image screen 46, and the subject information screen 146 may also be displayed on the display apparatus 30. In this case, the endoscopic image 40A, the past examination image screen 46, and the subject information screen 146 may be displayed adjacently, or the past examination image screen 46 and the subject information screen 146 may be displayed selectively in a manner allowing for comparison with the endoscopic image 40A.

Also, the endoscopic image 40A may be displayed on the touch panel display 44 of the tablet terminal 32. In this case, the endoscopic image 40A, the past examination image screen 46, and the subject information screen 146 may be displayed adjacently, or the past examination image screen 46 and the subject information screen 146 may be displayed selectively in a manner allowing for comparison with the endoscopic image 40A.

The endoscopic image 40A, the past examination image screen 46, and the subject information screen 146 may also be displayed on separate displays.

In the embodiment above, there is only one reference image 150, but this is merely one example, and there may also be multiple reference images 150. In this case, for example, multiple reference images 150 are selected from among the multiple examination result images 50 in the manner described above.

The embodiment above is described using an example in which the report 136 is stored in the NVM 126 of the server 78, but the technology of the present disclosure is not limited thereto. For example, the report 136 may also be stored in the NVM 98 of the endoscopic processing apparatus 22, the NVM 110 of the control apparatus 28, and/or a memory of the tablet terminal 32.

The embodiment above is described using an example in which the subject-identifying information 138 and the endoscopic examination information 140 are recorded in the report 136 and acquired from the report 136 by the processor 106, but the technology of the present disclosure is not limited thereto. At least part of the subject-identifying information 138 and at least part of the endoscopic examination information 140 may also be stored in the NVM 98 of the endoscopic processing apparatus 22, the NVM 110 of the control apparatus 28, and/or a memory of the tablet terminal 32. In this case, for example, the processor 106 may simply acquire at least part of the subject-identifying information 138 and at least part of the endoscopic examination information 140 from the NVM 98 of the endoscopic processing apparatus 22, the NVM 110 of the control apparatus 28, and/or a memory of the tablet terminal 32.

The embodiment above is described using an example of performing endoscope system processing using the endoscopic image 40 acquired by the camera 66, but the technology of the present disclosure is not limited thereto. For example, endoscope system processing using an ultrasound image obtained by endoscopic ultrasound may also be performed.

The embodiment above illustrates the endoscope 18 used with respect to the large intestine 88 as an example of a lower gastrointestinal endoscope, but this is merely one example, and the technology of the present disclosure is realized even with an endoscope used with respect to lower gastrointestinal organs other than the large intestine 88, an upper gastrointestinal endoscope, a cholangio-pancreatic endoscope, a bronchial endoscope, or the like.

The embodiment above is described using an example in which the location to be treated (cured, for example) inside the body of the subject 20 is identified in the second endoscopic examination, but the technology of the present disclosure is not limited thereto. For example, a location that was treated (cured, for example) in the past may also be identified to check the effect of the treatment (cure, for example) in the second endoscopic examination.

Checking the effect of the treatment (cure, for example) may involve, for example, checking the effect of a polypectomy location and/or checking an effect pertaining to a treatment to remove H. pylori.

The embodiment above is described using an example in which the endoscopic image display program 130 and the endoscope manipulation assistance program 132 are stored in the NVM 110 and the report management program 134 is stored in the NVM 126, but the technology of the present disclosure is not limited thereto. For example, the endoscope system program may also be stored in an SSD or a portable storage medium such as a USB memory. The storage medium is a non-transitory computer-readable storage medium. The endoscope system program stored in the storage medium is installed in the computers 80, 90, and/or 102. The processors 94, 106, and/or 122 execute the endoscope system program according to the endoscope system program.

In the embodiment above, the computers 80, 90, and/or 102 are illustrated by way of example, but the technology of the present disclosure is not limited thereto, and devices including an ASIC, an FPGA, and/or a PLD may also be applied in place of the computers 80, 90, and/or 102. A combination of a hardware configuration and a software configuration may also be used in place of the computers 80, 90, and/or 102.

The various types of processors indicated below can be used as hardware resources to execute the endoscope system processing described in the embodiment above. The processor may be, for example, a general-purpose processor that executes software, namely a program, to thereby function as hardware resources to execute the endoscope system processing. The processor may also be, for example, a special-purpose electronic circuit such as an FPGA, a PLD, or an ASIC, that is, a processor having a specially designed circuit configuration for executing specific processing. Any of these processors has a built-in or connected memory, and any of these processors uses the memory to execute the endoscope system processing.

The hardware resources to execute the endoscope system processing may be formed from one of these various types of processors, or may be formed from a combination of two or more processors of the same or different types (such as a combination of multiple FPGAs, or a combination of a processor and an FPGA). The hardware resources to execute the endoscope system processing may also be a single processor.

As a first example of a configuration using a single processor, a combination of one or more processors and software are used to form a single processor, and this processor functions as the hardware resources to execute the endoscope system processing. A second example is to use processor in which the functions of the entire system, including multiple hardware resources to execute the endoscope system processing, are realized by a single IC chip, as typified by an SoC. In this way, the endoscope system processing is realized by using one or more of the various types of processors above as hardware resources.

Furthermore, an electronic circuit combining circuit elements such as semiconductor elements can be used more specifically as the hardware structure of these various types of processors. Also, the endoscope system processing above is merely one example. Needless to say, unnecessary steps may be deleted, new steps may be added, and the processing sequence may be rearranged, insofar as the result does not depart from the gist of the technology of the present disclosure.

The descriptions and illustrations given above are detailed descriptions of portions related to the technology of the present disclosure, and are nothing more than examples of the technology of the present disclosure. For example, the above descriptions pertaining to configuration, function, action, and effect are descriptions pertaining to one example of the configuration, function, action, and effect of portions related to the technology of the present disclosure.

Needless to say, unnecessary portions may be deleted and new elements may be added or substituted with respect to the descriptions and illustrations given above, insofar as the result does not depart from the gist of the technology of the present disclosure. Also, to avoid confusion and to facilitate understanding of the portions related to the technology of the present disclosure, in the descriptions and illustrations given above, description is omitted in regard to common technical knowledge and the like that does not require particular explanation to enable implementation of the technology of the present disclosure.

In this specification, “A and/or B” is synonymous with “at least one of A or B”. That is, “A and/or B” means that: A only is a possibility; B only is a possibility; and a combination of A and B is a possibility. Also, in this specification, the same way of thinking as for “A and/or B” also applies when three or more matters are expressively linked using “and/or”.

All documents, patent applications, and technical standards mentioned in this specification are incorporated by reference herein to the same extent that individual documents, patent applications, and technical standards are specifically and individually noted as being incorporated by reference.

What is claimed is:

1. An information processing apparatus comprising:
a processor,
the processor configured to:
acquire a plurality of first endoscopic examination images from storage archiving the plurality of first endoscopic examination images, the plurality of first endoscopic examination images having been obtained by imaging a plurality of sites in a first endoscopic examination which is a previous or earlier endoscopic examination; and
cause a display apparatus to display at least one first endoscopic examination image satisfying a specific condition from among the plurality of first endoscopic examination images as a reference image in a period of carrying out a second endoscopic examination which is a current endoscopic examination, wherein:
the specific condition is a first condition, a second condition, or a third condition,
the first condition is a condition stipulating that a selection has been made according to an instruction accepted by an accepting apparatus with the plurality of first endoscopic examination images being displayed on a screen,
the second condition is a condition stipulating that a selection has been made by performing image recognition processing on the plurality of first endoscopic examination images and/or information processing on metadata of the plurality of first endoscopic examination images, and
the third condition is a condition stipulating that a selection has been made by performing image recognition processing on the plurality of first endoscopic examination images and/or information processing on metadata of the plurality of first endoscopic examination images, and that a selection has been made according to an instruction accepted by an accepting apparatus.

2. The information processing apparatus according to claim 1, wherein the processor is configured to cause the display apparatus to display the reference image in a timeout phase performed during the period of carrying out the second endoscopic examination.

3. The information processing apparatus according to claim 2, wherein the processor is configured to cause the display apparatus to display first information obtained during a period of carrying out the first endoscopic examination, in a timeout phase performed during the period of carrying out the second endoscopic examination.

4. The information processing apparatus according to claim 1, wherein the processor is configured to:
acquire second information from an apparatus storing the second information, the second information being required in a timeout performed during the period of carrying out the second endoscopic examination; and
cause the display apparatus to display the acquired second information.

5. The information processing apparatus according to claim 4, wherein the processor is configured to store the second information in the storage in a case in which the timeout performed during the period of carrying out the second endoscopic examination is completed.

6. The information processing apparatus according to claim 1, wherein information obtained in a timeout of the first endoscopic examination is associated with the first endoscopic examination images.

7. The information processing apparatus according to claim 1, wherein information obtained in a timeout of the second endoscopic examination is associated with a second endoscopic examination image obtained by imaging in the second endoscopic examination.

8. The information processing apparatus according to claim 1, wherein the metadata includes endoscopic examination information obtained during the period of carrying out the first endoscopic examination.

9. The information processing apparatus according to claim 8, wherein the endoscopic examination information includes information obtained in a timeout of the first endoscopic examination.

10. The information processing apparatus according to claim 1, wherein the processor is configured to perform first notification processing to provide a notification in a case in which a location corresponding to a characteristic portion in the reference image is shown in a second endoscopic examination image obtained by imaging in the second endoscopic examination.

11. The information processing apparatus according to claim 10, wherein the first notification processing includes processing to change a display appearance of the reference image.

12. The information processing apparatus according to claim 1, wherein the processor is configured to change a display appearance of the reference image according to a positional relationship between a characteristic portion in the reference image and a location which corresponds to the characteristic portion and which is shown in a second endoscopic examination image obtained by imaging in the second endoscopic examination.

13. The information processing apparatus according to claim 12, wherein the processor is configured to perform second notification processing to provide a notification in a case in which the positional relationship is a predetermined positional relationship.

14. The information processing apparatus according to claim 1, wherein the processor is configured to perform assistance processing to assist with making imaging conditions of the second endoscopic examination consistent with imaging conditions of the first endoscopic examination on the basis of the reference image and a second endoscopic examination image obtained by imaging in the second endoscopic examination.

15. The information processing apparatus according to claim 14, wherein the assistance processing includes output processing to output assistance information required to make the imaging conditions of the second endoscopic examination consistent with the imaging conditions of the first endoscopic examination.

16. The information processing apparatus according to claim 15, wherein the assistance information is derived on the basis of a result of comparing a first characteristic portion shown in the reference image with a second characteristic portion shown in the second endoscopic examination image.

17. The information processing apparatus according to claim 1, wherein the processor is configured to change the composition of the reference image according to a second endoscopic examination image obtained by imaging in the second endoscopic examination.

18. The information processing apparatus according to claim 1, wherein the processor is configured to cause the display apparatus to display the reference image and a second endoscopic examination image obtained by imaging in the second endoscopic examination in a manner allowing for comparison.

19. An endoscope apparatus comprising:
the information processing apparatus according to claim 1; and
an endoscope that images the plurality of sites in the endoscopic examinations.

20. An information processing method comprising:
acquiring a plurality of first endoscopic examination images from storage archiving the plurality of first endoscopic examination images, the plurality of first endoscopic examination images having been obtained by imaging a plurality of sites in a first endoscopic examination which is a previous or earlier endoscopic examination; and
causing a display apparatus to display at least one first endoscopic examination image satisfying a specific condition from among the plurality of first endoscopic examination images as a reference image in a period of carrying out a second endoscopic examination which is a current endoscopic examination, wherein:
the specific condition is a first condition, a second condition, or a third condition,
the first condition is a condition stipulating that a selection has been made according to an instruction accepted by an accepting apparatus with the plurality of first endoscopic examination images being displayed on a screen,
the second condition is a condition stipulating that a selection has been made by performing image recognition processing on the plurality of first endoscopic examination images and/or information processing on metadata of the plurality of first endoscopic examination images, and
the third condition is a condition stipulating that a selection has been made by performing image recognition processing on the plurality of first endoscopic examination images and/or information processing on metadata of the plurality of first endoscopic examination images, and that a selection has been made according to an instruction accepted by an accepting apparatus.
